(19) United States
(12) Patent Application Publication
(10) Pub. No.: US 2002/0148006 A1
(43) Pub. Date: Oct. 10, 2002

(54) Transgenic plants with modified sterol compositions

(75) Inventor: W. David Nes, Lubbock, TX (US)

Correspondence Address: Matthew L. Madsen, HOWREY SIMON ARNOLD & WHITE, LLP, 750 Bering Drive, Houston, TX, 77057-2198, US

(73) Assignee: Asgrow Seed Company, L.L.C.

(21) Appl. No.: 09/779,144

(22) Filed: Feb. 8, 2001

Related U.S. Application Data
(62) Division of application No. 09/106,926, filed on Jun. 29, 1998.
Application No. 09/106,926 is a continuation-in-part of application No. 08/998,339, filed on Dec. 24, 1997, now abandoned.
(60) Provisional application No. 60/033,923, filed on Dec. 26, 1996.

(51) Int. Cl. A01H 5/00; C07H 21/04; C12N 9/02

(52) U.S. Cl. 800/281; 435/189; 435/52; 536/23.2

(57) ABSTRACT

Plant phytosterol compositions are modulated in transgenic plants to confer resistance to insects, nematodes, fungi, and/or other environmental stresses, and/or to improve the nutritional value of the plants. Recombinant DNA molecules of the invention encode RNA or protein sequences capable of altering plant sterol profiles by affecting the expression or activity of sterol biosynthetic enzymes. The DNA molecules are transformed into plant cells and plants having altered sterol compositions are regenerated therefrom.

This application is a continuation-in-part application of U.S. application Ser. No. 08/998,339, filed Dec. 24, 1997.

### FIELD OF THE INVENTION

The present invention broadly relates to plant genetic engineering. More particularly, it concerns the manipulation of the levels and/or activities of endogenous plant phytosterol compositions as a strategy for minimizing crop damage due to plant insects and other pests, and/or for improving the nutritional value of plants.

### BACKGROUND OF THE INVENTION

Productivity in agricultural industries can be adversely affected by various environmental stresses, including drought, severe cold, weeds, and organisms that feed on crops. Conventional approaches for alleviating weeds and parasitic organisms have relied almost exclusively on chemical herbicides, pesticides and fungicides. Widespread use of these agrochemicals, however, has led to development of resistance. In fact, insect resistance has been reported against most major classes of insecticides including organophosphates, chlorinated hydrocarbons, and carbamates.

Sterols comprise a class of essential natural compounds required to some extent by all eukaryotic organisms. They have a common tetracyclic steroid nucleus and a side chain, as shown in the diagram below. Some sterols serve a structural role in cell membranes, while others are required during development.

Plants produce more than 250 different phytosterols (Akisha et al., 1992). As many as 60 sterols have been identified in the single species, Zea mays (corn) (Guo et al., 1995). However, insects, fungi and nematodes, as well as many other sterol-less parasitic organisms, do not synthesize all of their necessary sterols de novo. Rather, they satisfy their nutritional requirements for sterols by feeding on plants. This fact has been utilized in the development of commercial agrochemicals such as triazoles, pyrimidines and azasterols, which act by interfering with production of sterols within parasitic organisms.

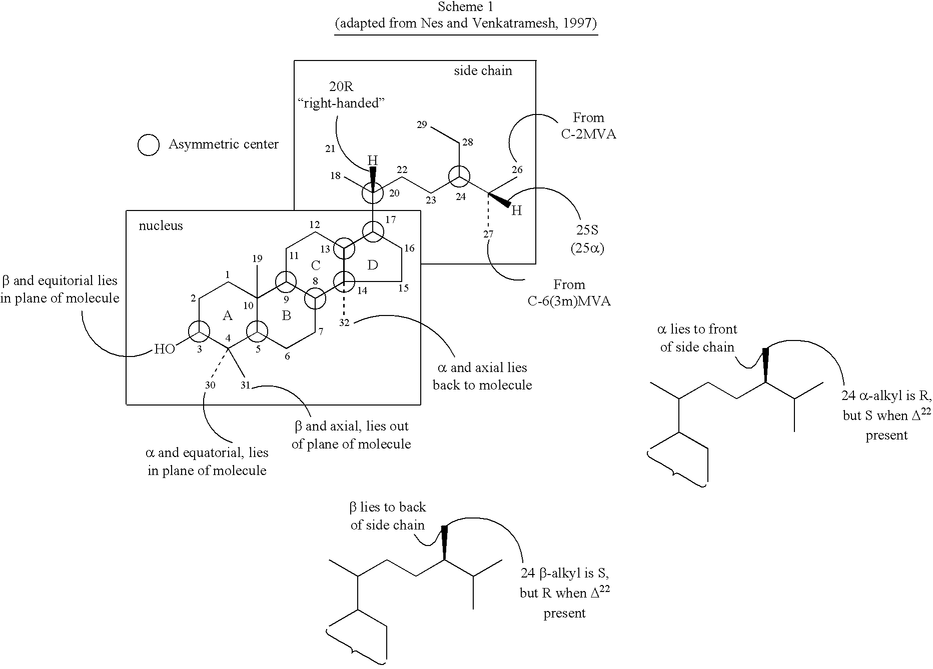

Recent advances in molecular biology have made it possible to introduce advantageous traits into plants via genetic engineering. Some forms of insect resistance have been introduced into plants by genetic approaches. For example, transgenic plants expressing foreign genes encoding endotoxins of Bacillus thuringiensis (Bt) can confer on the plants the ability to kill pests which feed on them. Unfortunately, approaches such as this are effective only against the particular insects susceptible to the endotoxin. There remains in the agricultural industries a continual need for alternative pest control strategies, particularly those that could be broadly effective against numerous pests/pathogens.

### SUMMARY OF THE INVENTION

The present invention broadly relates to approaches for genetically engineering plants to have altered sterol compositions, levels and/or metabolism. Such approaches can increase the plants natural insect resistance, can increase the plants resistance to drought and cold, and/or can improve the nutritional/health value of the plants.

In accordance with one aspect of the invention, there are provided recombinant DNA molecules comprising:

a promoter which functions in plants to cause the production of an RNA sequence, operably linked to

a DNA coding sequence encoding an enzyme which binds a first sterol and produces a second sterol, operably linked to

a 3′ non-translated region which causes the polyadenylation of the 3′ end of the RNA sequence; wherein the promoter is heterologous with respect to the DNA sequence.

The DNA coding sequence encoding an enzyme which binds a first sterol and produces a second sterol can be in the sense or antisense orientation. Thus, the DNA molecule of the invention can encode a non-translatable RNA molecule (e.g., antisense or cosuppression) or a protein molecule. The RNA or protein so produced selectively targets the expression and/or activity of a sterol biosynthetic enzyme to affect a desired change in the phytosterol profile of the plant.

Therefore, in accordance with another aspect of the present invention, there is provided an approach for modifying the sterol composition of plants to increase their resistance to insects, nematodes, and pythiaceous fungi. This aspect of the invention enhances the plant's ability to resist pests and disease by modifying the composition and/or distribution profile of certain phytosterols. Such an approach overcomes many of the limitations inherent in the use of agrochemicals, or with transgenic plants where the foreign product introduced into the plant has the potential to eventually select for new mechanisms of resistance by the pest. The present invention retains the benefits obtained through the use of agrochemicals, but avoids many of their disadvantages. By targeting an existing essential pathway in pests and pathogens, this invention reduces the likelihood of the evolution of mechanisms which circumvent this pathway.

Plant sterol composition is modified in this aspect by increasing the amount of non-utilizable sterols such as 4, 4-dimethyl sterols, 4-methyl sterols, 9β,19-cyclopropyl sterol, Δ⁷-sterol, Δ⁸-sterol, 14α-methyl sterol, Δ²³⁽²⁴⁾-24-alkyl sterol, Δ²⁴⁽²⁵⁾,24-alkyl sterol or Δ²⁵⁽²⁷⁾,24-alkyl sterol. Alternatively, sterol compositions can be modified to contain lower levels of sterols having a Δ⁵ group.

Another aspect of the present invention relates to producing sterols in plants that confer resistance to drought and cold in plants.

Another aspect of the invention relates to altering the sterol profile of plants such that levels of cholesterol-lowering sterols are increased.

The aspects of the invention described herein are typically achieved by modifying the expression and/or activities of sterolic enzymes, preferably S-adenosyl-L-methionine-Δ²⁴-sterol methyl transferases (SMT_{I} and SMT_{II}), C-4 demethylase, cycloeucalenol to obtusifoliol-isomerase, 14α-methyl demethylase, Δ⁸ to Δ⁷-isomerase, Δ⁷-sterol-C-5-desaturase, or 24,25-reductase.

Another aspect of the invention is directed to transgenic plants having altered levels of selected sterols, produced by introducing recombinant DNA molecules of the invention into the genome of plant cells and selecting for cells expressing said molecule. Transgenic plants are regenerated from the transformed plant cells and plants containing the recombinant DNA are grown to maturity. Plants expressing the recombinant DNA are identified and those having a desired sterol profile in accordance with the present invention are selected and propagated.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows HPLC radiocount (panel B) and mass spectrum (panel A) results of testing SMT enzyme with radiolabeled substrate coenzyme;

FIG. 2 shows six inhibitors used to test the SMT enzyme;

FIG. 3 shows SMT activity during seedling development;

FIG. 4 shows the pathway of sterol end-products during development of seedlings;

FIG. 5 shows the yeast SMT gene sequence (panel B; SEQ ID NO: 1) and the deduced amino acid sequence (panel A; SEQ ID NO:2) with the predicted conserved regions highlighted;

FIG. 6 shows the Arabidopsis SMT gene (panel B; SEQ ID NO:3) and deduced amino acid (panel A; SEQ ID NO:4) sequences;

FIG. 7 shows the ERG6 constructs prepared with pUC18cpexp expression cassette;

FIG. 8 shows sequences of yeast SMT gene (SEQ ID NO:5). Underlined sequences are those used as primers for screening genomic DNA from transgenic tomato plants; and

FIG. 9 shows structures of plant sterols tested on Heliothis zea and found to be utilizable or non-utilizable.

FIG. 10 (SEQ ID NO:6) shows the nucleotide and amino acid sequences of the corn SMT gene.

### DESCRIPTION OF ILLUSTRATIVE EMBODIMENTS

Phytosterols

The phytosterol metabolic pathway consists of enzymes that act on the tetracyclic ring nucleus and the side chain. The major pathway in advanced vascular plants starts from cycloartenol (I):

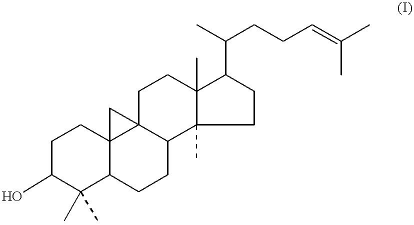

and ends with Δ⁵-24-alkyl sterols, predominantly sitosterol (II), stigmasterol (III) and campesterol (IV):

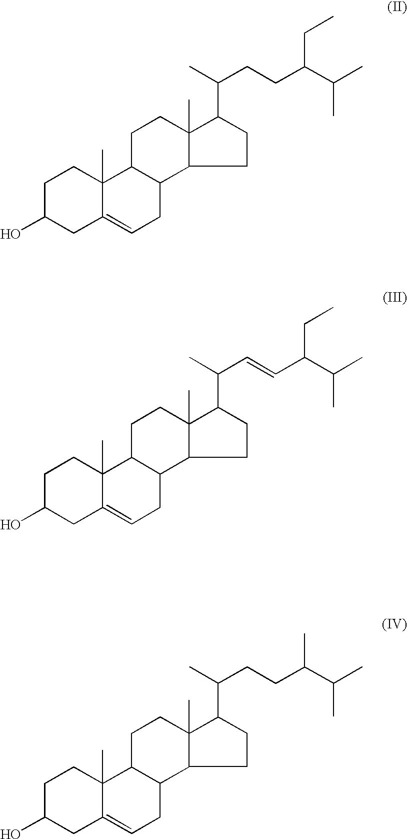

The number of alternate pathways is sufficiently great to produce as many as 60 or more different sterols in a single plant. These alternate pathways vary according to tissue- and development-specific genetic programs.

Studies of sterol metabolism have utilized inhibitors of sterol biosynthesis. These inhibitors include several commercial fungicides which block sterol metabolic pathways in plant pathogenic fungi and thereby inhibit their growth. The following steps of the major metabolic pathway were determined using metabolic inhibitors. The major pathway consists of the 12 chemical transformations as follows.

In reaction 1, the enzyme S-adenosyl-L-methionine-sterol-C-24 methyl transferase (SMT_{I}) catalyzes the transfer of a methyl group from a coenzyme, S-adenosyl-L-methionine, to the C-24 center of the sterol side chain. The circled sterol feature is the functional group undergoing transformation.

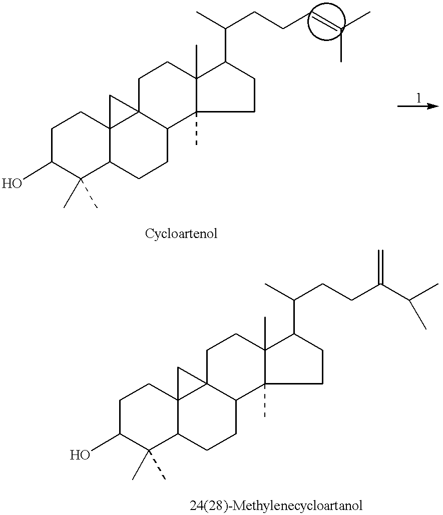

This is the first of two methyl transfer reactions, and is an obligatory and rate-limiting step of the sterol-producing pathway in plants. A different SMT enzyme, SMT_{II}, catalyzes the conversion of cycloartenol to a Δ²³⁽²⁴⁾-24-alkyl sterol, cyclosadol (Guo et al., 1996).

Reaction 2 involves a demethylation at C-4. This is the first of several demethylation reactions in the nucleus.

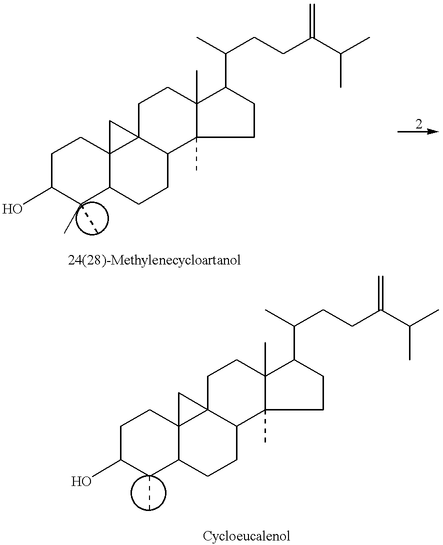

Reaction 3 involves opening the cyclopropyl ring at C-9(10) by the enzyme cycloeucalenol-obtusifoliol isomerase (COI), which also creates a double bond at C-8.

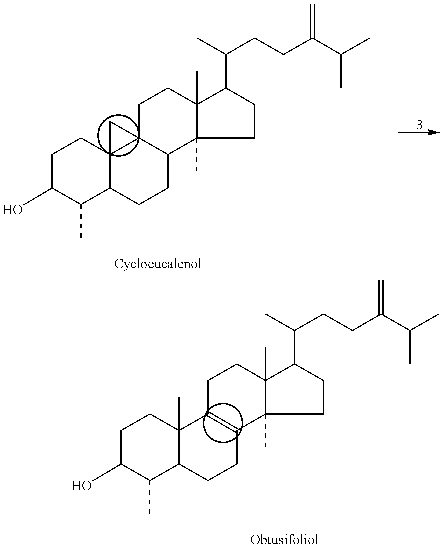

Reaction 4 involves a demethylation at C-14 which removes the methyl group at C-14 and creates a double bond at C-14.

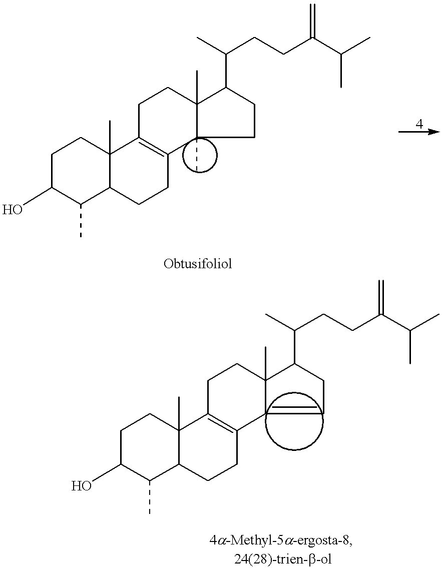

Reaction 5 is catalyzed by a Δ¹⁴ reductase.

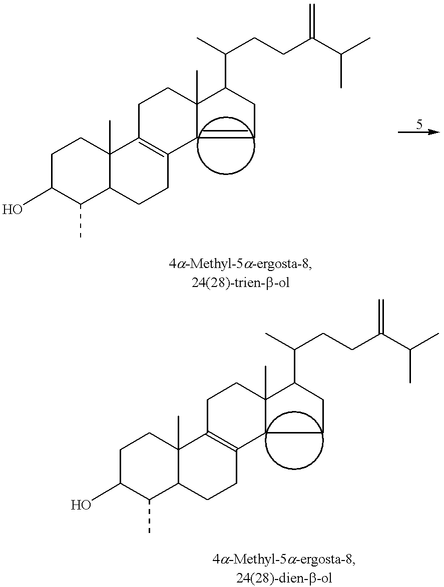

Reaction 6 involves a Δ⁸- to Δ⁷-isomerase reaction which produces a Δ⁷ group.

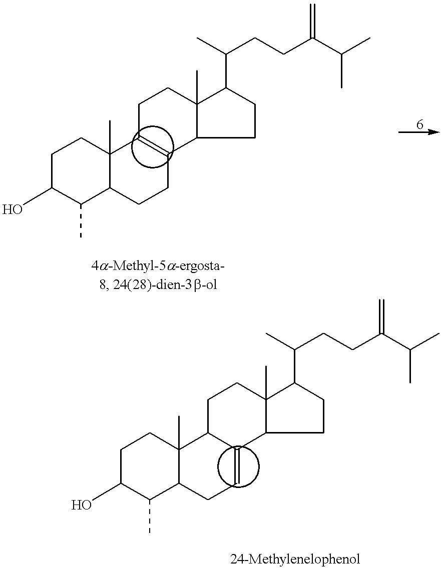

Reaction 7 is a second C-methylation of the sterol side chain. The reaction is catalyzed by SMT_{I}, the same enzyme that initiated the major pathway (Tong et al., 1997).

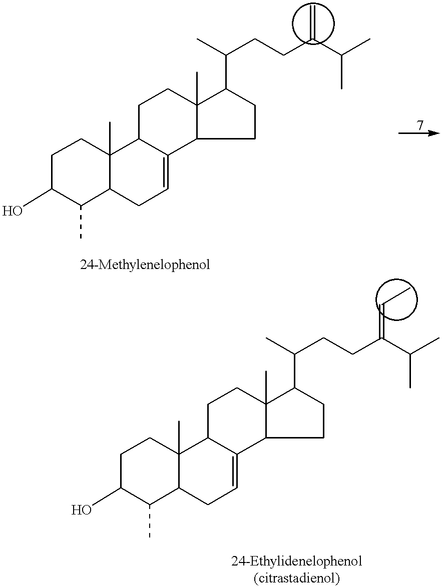

Reaction 8 involves a C-4 demethylase to generate a 4,4-desmethyl sterol.

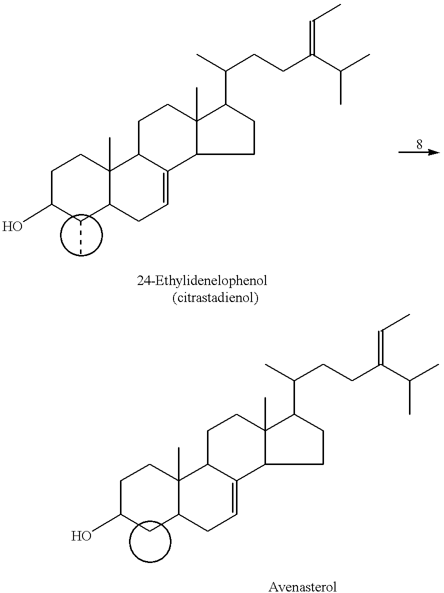

Reaction 9 involves a Δ⁵ desaturase, producing a

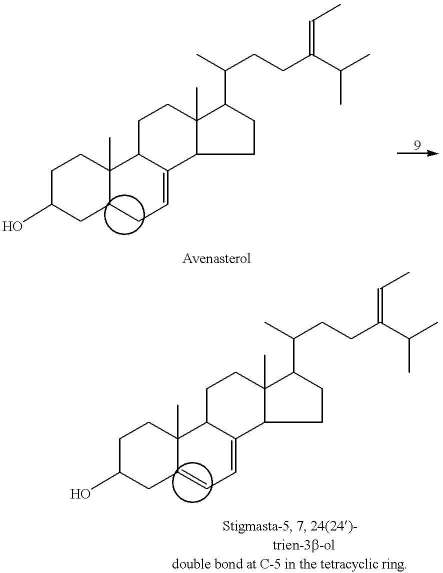

The product of reaction 9 is then transformed in reaction 10 by a Δ⁷-reductase by removing the double bond at C-7.

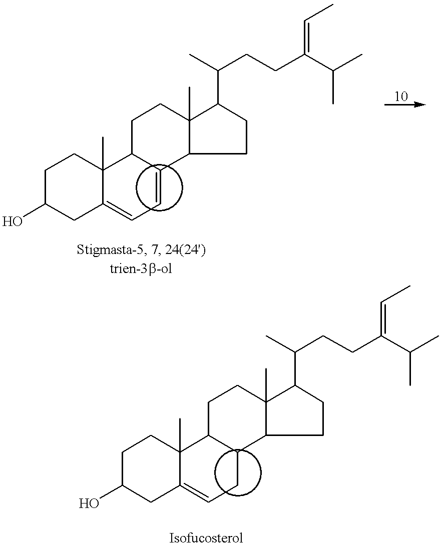

Reaction 11, involves a Δ²⁴⁽²⁸⁾- to Δ²⁴⁽²⁵⁾-isomerase which modifies the side chain. (It is believed that this reaction would have proceeded from the product of reaction 5 if the kinetics were more favorable.)

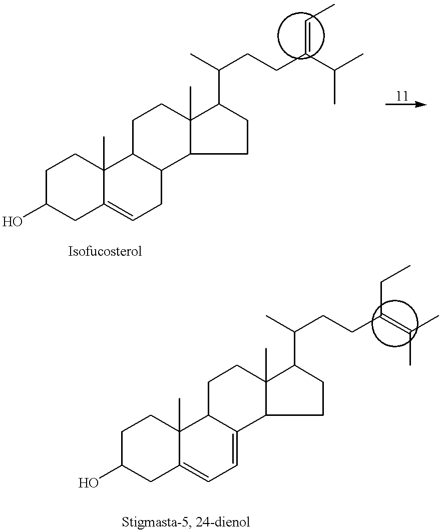

Reaction 12: the Δ²⁴⁽²⁵⁾ double bond at C-24 is reduced stereoselectively to produce sitosterol (II).

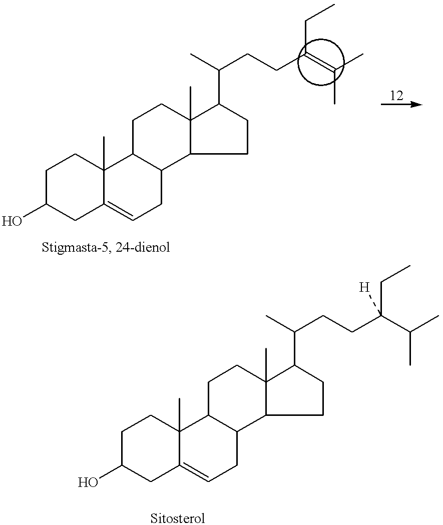

In addition to this major pathway of sterol biosynthesis, it has been found that a developmental program regulates expression of the SMT enzymes. In corn, enzymology studies have shown that two different SMT enzymes exist (SMT_{I} and SMT_{II}) whose expression depends on the tissue and stage of differentiation. Blades mainly contain 24-ethyl sterols (resulting from the activity of SMT_{I}), whereas the sheaths contain mainly 24-methyl sterols (VI) (resulting from the activity of SMT_{II}). These sterols are the products of the two different SMT enzymes that react with the same starting material, cycloartenol (Guo et al., 1995 and 1996).

The first enzyme, SMT_{I}, produces Δ²⁴⁽²⁸⁾-methylene and the second enzyme produces Δ²³⁽²⁴⁾-methyl sterol (V). The first type of SMT enzyme leads to a utilizable sterol (a sterol which can be utilized by insects, pythiaceous fungi, and nematodes to complete their life cycles). The second type of SMT enzyme produces a non-utilizable sterol (a sterol which cannot be utilized by insects, pythiaceous fungi, and nematodes to complete their life cycles) (Nes et al., 1997). Therefore, one could inhibit expression of the first type of SMT enzyme so as to cause accumulation of the non-utilizable Δ²³⁽²⁴⁾-methyl sterols.

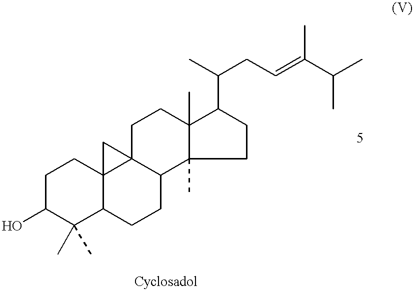

As a result, the sterols that accumulate in the tissue contain a double bond at C-23 (VI) and a methyl at C-24.

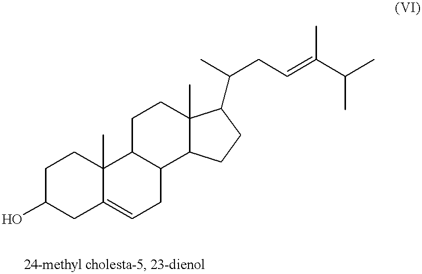

Recombinant DNA Molecules:

In order to achieve a desired alteration in sterol composition, the invention provides recombinant DNA molecules for use in the production of transgenic plants. A recombinant DNA molecule of the invention generally comprises a promoter region capable of causing the production of an RNA sequence in plants, a structural DNA sequence, and a 3′ non-translated region.

Transcription of DNA into mRNA is regulated by the region of a gene referred to as the “promoter”. The promoter region contains a sequence of bases that signals RNA polymerase to associate with the sense and antisense DNA strands and to use the sense strand as a template to make a corresponding strand of mRNA complimentary to the sense DNA strand. This process of mRNA production using a DNA template is commonly referred to as gene “expression” or “transcription”.

In the recombinant DNA molecules of the invention, it is generally preferred that the promoter is heterologous with respect to the DNA coding sequence. The term “heterologous” with respect to a promoter means that the DNA coding sequence of a recombinant DNA molecule of the invention is not derived from the same gene to which the promoter is attached.

Promoters may be obtained from a variety of sources, such as plants and plant viruses. The particular promoters selected for use in embodiments of the present invention should preferably be capable of causing the production of sufficient expression to affect the desired change in the sterol distribution profile of the plant.

A number of promoters which are active in plant cells have been described in the literature, and are suitable for use in the DNA molecules of this invention. These include, for example, the cauliflower mosaic virus (CaMV) 35S promoter (Odell et al., 1985), the Figwort mosaic virus (FMV) 35S (Sanger et al., 1990), the sugarcane bacilliform virus promoter (Bouhida et al., 1993), the commelina yellow mottle virus promoter (Medberry and Olsewski 1993), the light-inducible promoter from the small subunit of the ribulose-1,5-bis-phosphate carboxylase (ssRUBISCO) (Coruzzi et al., 1984), the rice cytosolic triosephosphate isomerase (TPI) promoter (Xu et al., 1994), the adenine phosphoribosyltransferase (APRT) promoter of Arabidopsis (Moffatt et al., 1994), the rice actin 1 gene promoter (Zhong et al., 1996), the mannopine synthase and octopine synthase promoters (Ni et al., 1995). All of these promoters have been used to create various types of DNA constructs which have been expressed in plants.

Recombinant DNA molecules also typically contain a 5′ non-translated leader sequence. This sequence can be derived from the promoter selected to express the gene, and if desired, can be specifically modified so as to increase translation of the mRNA. The 5′ non-translated regions can also be obtained from viral RNAs, from suitable eukaryotic genes, or from synthetic gene sequences.

The structural DNA sequence of the recombinant DNA molecule of the invention will cause the desired alteration in the sterol profile of the plant, as discussed further below.

The 3′ non-translated region of a recombinant DNA molecule of the invention can be obtained from various genes which are expressed in plant cells. For example, the nopaline synthase 3′ untranslated region (Fraley et al., 1983), the 3′ untranslated region from pea ssRUBISCO (Coruzzi et al., 1994), and the 3′ untranslated region from soybean 7S seed storage protein gene (Schuler et al., 1982) are frequently used. The 3′ non-translated region of a recombinant DNA molecules contains a polyadenylation signal which functions in plants to cause the addition of adenylate nucleotides to the 3′ end of the RNA.

Other desired regulatory sequences known to the skilled individual, or combinations thereof, can be included in a recombinant DNA molecule of the invention. For example, intron sequences are frequently included in recombinant DNA molecules used for producing transgenic plants in order to enhance expression levels. Examples of plant introns suitable for expression in plants can include maize hsp70 intron, rice actin 1 intron, maize ADH 1 intron, Arabidopsis SSU intron, Arabidopsis EPSPS intron, petunia EPSPS intron and others known to those skilled in the art.

Plant Transformation and Regeneration

A double stranded DNA molecule of the present invention can be inserted into the genome of a plant by any suitable method. Numerous plant transformation methods have been described, including Agrobacterium-mediated transformation, the use of liposomes, electroporation, chemicals that increase free DNA uptake, free DNA delivery via microprojectile bombardment, transformation using viruses or pollen, etc.

After transformation of cells (or protoplasts), choice of methodology for the regeneration step is not critical, with suitable protocols being available for hosts from Leguminosae (alfalfa, soybean, clover, etc.), Umbelliferae (carrot, celery, parsnip), Cruciferae (cabbage, radish, rapeseed, etc.), Cucurbitaceae (melons and cucumber), Graminae (wheat, rice, corn, etc.), and Solanaceae (potato, tobacco, tomato, peppers). Methods for transformation and regeneration of dicots, primarily by use of Agrobacterium tumefaciens, and obtaining transgenic plants, have been described for numerous plant species, including cotton (U.S. Pat. Nos. 5,004,863; 5,159,135; 5,518,908), soybean (U.S. Pat. Nos. 5,569,834; 5,416,011; Christou et al. (1988)), Brassica (U.S. Pat. No. 5,463,174), peanut (Cheng et al. (1996); papaya (Yang et al. (1996), and pea (Schroeder et al. (1993); De Kathen and Jacobsen (1990)), and others.

Transformation of monocots using electroporation, particle bombardment, and Agrobacterium have also been reported. Transformation and plant regeneration have been achieved, for example, in asparagus (Bytebier et. (1987)), barley (Wan and Lemaux (1994)), maize (Rhodes et al. (1988); Gordon-Kamm et al. (1990); Fromm et al. (1990); Koziel et al. (1993);

Armstrong et al. (1995)), oat (Somers et al. (1992)), orchardgrass (Horn et al. (1988)), rice (Toriyama et al. (1988); Battraw and Hall (1990); Christou et al. (1991)), rye (Bryant (1987)), sugar cane (Bower and Birch (1992)), tall fescue (Wang et al. (1992)), and wheat (Vasil et al. (1992); Weeks et al. (1993)).

For reviews of plant transformation and/or regeneration methodologies see, for example, Ritchie and Hodges (1993) or Hinchee et al. (1994).

Insect/Pest Resistance via Phytosterol Alterations

A series of phytosterols were tested in insects and many were found to be unable to support insect growth, i.e., were non-utilizable. These sterols included 9,19-cyclopropyl sterols.

Furthermore, novel Δ⁽²³⁽²⁴⁾- and Δ²⁴⁽²⁵⁾-alkene and Δ²⁵⁽²⁷⁾-alkyl sterols were also determined to be unable to support insect growth and maturation. These were tested in vivo using Heliothis zea (a corn earworm), cultured on synthetic media that was sterol-free with the exception of added test sterols. It was found that if the ratio of utilizable to nonutilizable sterols was 1:9 or less, insects could not undergo normal develop. In fact, even at 1:1 ratios, insect development was adversely affected (Nes et al., 1997).

The metabolism of insects, nematodes and pythiaceous fungi is limited by the availability of major plant sterols (Nes et al., 1982 and 1997). These pests cannot use a sterol with a C-4 methyl group; a 9β, 19-cyclopropyl group, or a Δ⁸ group. Furthermore, nematodes and insects cannot utilize 14-α methyl-sterols, and some insects, including lepidoptera, diptera and coleoptera, cannot utilize C-24 alkyl sterols with Δ²⁴⁽²⁵⁾, Δ²³⁽²⁴⁾, or Δ²⁵⁽²⁷⁾ groups for mechanistic reasons. Some insects cannot utilize sterols lacking a Δ⁵ group. Consequently, elevation of these sterols in plants would provide a detrimental dietary source of sterols for these pests.

The DNA molecule of the present invention, when expressed in transgenic plants, will cause alterations in the composition/distribution of the sterols present in the plant. In one preferred embodiment, the DNA molecule causes the accumulation of sterols that are non-utilizable by insects and other pests, so as to increase the plants resistance to the organisms. This can be accomplished, for example, by a number of approaches, including overexpression, antisense, cosuppression etc. The DNA molecule of the invention will typically target an endogenous gene encoding an enzyme selected from the kinetically favored pathways of sterol biosynthesis.

In this embodiment, it is preferred that gene expression and/or translation of a sterol biosynthetic enzyme is targeted for inhibition. This inhibition can be achieved, for example, by engineering a DNA molecule of the invention to produce an antisense, ribozyme or cosuppression RNA molecule complementary to an endogenous gene being targeted. Approaches for the targeted inhibition of gene expression are well known to the skilled individual (for reviews, see Bird et al., 1991; Schuch, 1991; Gibson et al., 1997)

A preferred target for inhibition is the S-adenosyl-L-methionine-Δ²⁴⁽²⁵⁾-sterol methyl transferase (SMT) enzyme because it is now known to represent the critical slow step in phytosterol transformations (Nes and Venkatramesh, 1997). By targeting this sterol gene with an antisense or cosuppression construct, expression of SMT enzyme can be effectively suppressed, thereby causing the accumulation of non-utilizable sterols.

Besides SMT, other genes in the phytosterol transformation pathway can also be targeted in this and other embodiments of the invention in order to alter the profile of sterols in transgenic plants. The preferred target will depend on the application, however the approach is the same, i.e., to express an RNA or protein molecule capable of modifying the sterol composition of the plant in a desirable manner.

Therefore, in addition to SMT, other preferred cellular targets for causing sterol modifications include:

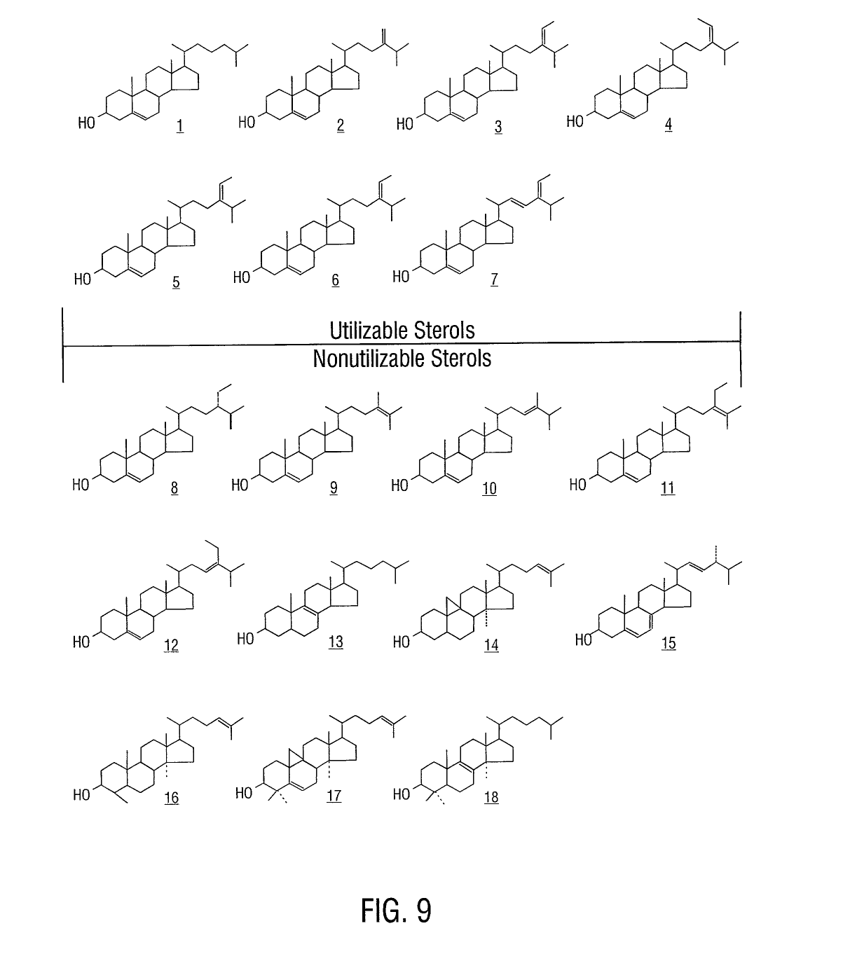

(i) C-4 demethylase: This enzyme is involved in the removal of the two methyl groups at C-4 and represents reactions 2 and 8 in the description section. A single protein is responsible for both the reactions. Blocking this enzyme will lead to accumulation of 4,4-dimethyl sterols such as cycloartenol, 24(28)-methylene cycloartenol or a novel sterol such as 24-dihydrolanosterol (structure 18 in FIG. 9). All these are nonutilizable sterols. This may be achieved through suppression of this gene in plants.

(ii) Cycloeucalenol to obtusifoliol isomerase (COI) and Δ⁸-to-Δ⁷ isomerase: These enzymes represent reactions 3 and 6 in the pathway. Certain fungicides are known to block these two enzymes in plants leading to the accumulation of 9β,19-cyclopropyl sterols. Locusts reared on these treated plants are known to have abnormal development and levels of cholesterol and ecdysteroids in these insects are depleted. This suggests that if either of these enzymes are disrupted or suppressed, the plant sterols can be altered such that they will not support insect development (Coste et al., 1987).

(iii) C-14 demethylase: This is reaction 4 in the pathway. There are several fungicides and plant growth regulators that block this step in fungi and plants. In plants this blockage leads to a depletion of the normal Δ⁵-sterols and an accumulation of 9β,19-cyclopropyl, 14α-methyl and Δ⁸-sterols that are intermediates of the main phytosterol pathway. These are also non-utilizable sterols. Studies with chemical inhibitors have also shown that plants accumulating these intermediates are tolerant to water and cold stress. Thus, suppression of this enzyme activity through gene manipulation is also a useful strategy.

(iv) Δ⁷-sterol-C-5-desaturase: This is reaction 9 in the pathway. Inhibition of this enzyme leads to a depletion of Δ⁵-sterols and an increase in Δ⁷ sterols. Certain insects are known to be unable to metabolize Δ⁷-sterols into ecdysteroids. Therefore, accumulation of Δ⁷-sterols in plants can also provide a way to form non-utilizable sterols. Further, A sterols can replace Δ⁵-sterols in plant membranes without any morphological changes in plant development.

(v) C-24 reductase: This is a terminal step in phytosterol transformation (reaction 12) during the formation of sitosterol, the major Δ⁵-sterol in plants. Disruption or suppression of the gene encoding this enzyme would result in the accumulation of Δ²⁴⁽²⁵⁾-24-alkyl sterols which are also non-utilizable.

Many of the genes encoding these preferred sterol biosynthetic enzymes to be targeted by the present invention have been isolated from yeast (for review, see Lees et al., 1997). Some have been isolated from plants. For example, SMT genes have been isolated from soybean (Shi et al., 1996), Arabidopsis (Husselstein et al., 1996; Bouvier-Nave et al., 1997) tobacco and castor (Bouvier-Nave et al., 1997); and corn (Grabenok et al., 1997). Other plant sterol biosynthetic genes that have been isolated include delta7-sterol-C5-desaturase from Arabidopsis (Gachotte et al., 1996) and cycloartenol synthase from Arabidopsis (Corey et al., 1993).

Where not available, the gene encoding a sterol biosynthetic enzyme can be readily isolated from a desired source by approaches known to the skilled individual. For example, an isolated gene or cDNA from one source can be used as a hybridization probe for the isolation of homolgous sequences from other sources. However, it should be noted that a DNA molecule of the invention should be active in numerous plant types, regardless of the source of the sterol biosynthetic gene used in the targeting construct, given the successful demonstration provided herein of using a yeast ERG6 antisense construct to alter the sterol profile in tomato.

Preferably, the following sterolic metabolic enzymes are targeted for inhibition: S-adenosyl-L-methionine-Δ²⁴-sterol methyl transferase, C-4 demethylase, cycloeucalenol to obtusifoliol-isomerase, 14α-methyl demethylase, Δ⁸- to Δ⁷-isomerase, Δ⁷-sterol-C-5-desaturase, or a 24,25-reductase.

Plants produced according to this embodiment preferably have increased amounts of certain sterols that are non-utilizable, particularly 4-methyl sterol, 9β,19-cyclopropyl sterol, Δ⁸-sterol, Δ⁷-sterol 14α-methyl sterol, Δ²³⁽²⁴⁾, 24 sterol, Δ²⁴⁽²⁵⁾-24-alkyl sterol or Δ²⁵⁽²⁷⁾-24-alkyl sterol, or decreased levels of sterols having a Δ⁵ group.

Preferred crops for use in providing insect resistance according to this embodiment of the invention include corn (European corn borer, corn earworm, fall armyworm), rice, sorghum, forestry, potato, tomato (tomato hornworm), and vegetable brassicas.

Preferred crops for use in providing nematode resistance according to this embodiment of the invention include soybean (soybean cyst nematode), tomato (root knot nematode), sugarbeet and cucurbits.

Preferred crops for use in providing fungal resistance according to this embodiment of the invention include corn, rice, wheat, sorghum, soybean (Phytophthora root rot), sunflower, forestry, fruits and berries, potato (late blight), tomato (late blight), sugarbeet, cucurbits, and vegetable brassicas.

Phytosterols as Cholesterol-lowering Agents

Animal and human studies have demonstrated that phytosterols can reduce serum and/or plasma total cholesterol and low density lipoprotein (LDL) cholesterol (Ling and Jones, 1995). In this regard, transgenic plants having altered sterol profiles could be instrumental in establishing a dietary approach to cholesterol management and cardiovascular disease prevention.

Structure-specific effects of individual phytosterols have recently been shown where saturated phytosterols, such as sitostanol, are more efficient compared to unsaturated compounds such as sitosterol in reducing cholesterol levels. Another structural feature that seems to play a role is esterification of the phytosterols. Some studies suggest that the ferrulate esters of sitosterol, sitostanol or cycloartenol have a more potent effect on lowering serum cholesterol than the corresponding free sterols (Meittinen and Vanhanen, 1994).

Some of the natural sources of phytosterols in the diet are rice bran oil, corn fiber oil and soybean oil. Rice bran and corn fiber are by far the most enriched sources of phytosterols.

Soybean phytosterols are a byproduct of the oil refining process. Technologies that can generate higher levels of these nutritionally useful phytosterols in these and other plants will assist in the development of new food products to improve human health and wellness.

Therefore, the present invention, in another embodiment, relates to increasing cholesterol-lowering sterols in transgenic plants. For example, with a recombinant DNA molecule of the invention, the conversion of cycloartenol in developing seeds can be inhibited, for example by antisense, cosuppression, or ribozyme-mediated inhibition of SMT expression, thereby leading to an accumulation of this sterol in seed oils. Alternatively, the SMT gene can be overexpressed in order to increase the levels of sitosterol.

Preferred crops for use in accordance with this embodiment of the invention include sunflower, corn, soybean, oilseed brassicas and cotton.

Stress Tolerance through Alterations in Phytosterols

Another embodiment of this invention derives from the fact that certain sterols are associated with reducing water permeability of membranes. For this reason, sterol manipulation should provide an effective means for preventing or at least minimizing drought induced damage. Several studies with chemical inhibitors of sterol biosynthesis have documented that the treated plants show secondary physiological responses that include tolerance to environmental stresses such as drought and frost (Fletcher, 1988). Such responses are primarily due to elevated levels of hormones such as abscisic acid. However, changes in membrane fluidity have also been recognized as being responsible for stress tolerance (Steponkus, 1984).

Membrane fluidity is controlled by several factors such as the type of sterols and fatty acids and the ratio between fatty acids and sterols in the membranes. Of these factors, the type of sterols is by far the most important factor. A principal function of the sterols is to buffer membranes against abrupt changes in fluidity. They also may have more specific influences on the activity of membrane-bound enzymes. An impairment of sterol biosynthesis, through the application of inhibitors, resulting in depletion of terminal sterols and accumulation of intermediates might therefore be expected to alter membrane function.

There is evidence to show that inhibition of sterol biosynthesis in plants leads to elevated levels of abscisic acid and closure of stomata (Haeuser, C. et al 1990 J. Plant Physiol. 137: 201-207). How this process is mediated is not clear. But what is well documented is that modification of phytosterols can lead to some forms of stress tolerance, which is most likely mediated by elevated levels of abscisic acid. Further, in all these studies with chemical inhibitors of sterol biosynthesis, the accumulating sterols are those recognized in this invention as nonutilizable. These are again, 9β,19-cyclopropyl sterols, 14α-methyl sterols and Δ⁸-sterols. Thus, formation of non-utilizable sterols in plants through the various gene manipulation strategies described in this invention will not only protect the plants from pests and pathogens but also from environmental stresses such as drought and cold. Preferred sterols to be elevated in this aspect include Δ⁵-24 alkyl sterols, such as 24-methyl cholesta-5,23-dienol, and cycloartenol.

Preferred crops for use in accordance with this embodiment of the invention include corn, wheat, rice, sorghum, soybean, oilseed brassicas (rapeseed, canola), sunflower, palm, peanut, cotton, forestry, fruits, berries, nuts, potato, tomato, sugarbeet, sugarcane, cucurbits (squash, melons, cucumbers, watermelons, pumpkins), vegetable brassicas, alfalfa, ornamental crops, turfgrass, peanut, tea and coffee.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention. Unless specifically indicated, all techniques discussed in the description above and used in the examples which follow can be performed by standard molecular biological and biochemical methodologies well known to the skilled individual (as described, for example, in Sambrook et al., 1989).

### EXAMPLES

### Example 1

### Plant Phytosterols

Sterol isomers were extracted from corn and were isolated to homogeneity using chromatographic methods. Novel phytosterols were identified with side chains that have been found to be non-utilizable in insects.

The sterols were structurally characterized by mass spectroscopy and ¹H and ¹³C nuclear magnetic resonance (NMR) (Table 1) (Guo et al, 1995).

The initial studies showed that 4-day corn shoots could produce mono- and di-alkylated sterols at C-24. Corn could produce those sterols, since isolated 24(28)-methylene and 24(28)ethylidene sterols were obtained from seedling tissue of corn and their structures were confirmed by mass and proton nuclear magnetic resonance spectroscopy.

TABLE 1
Sterol Composition of Zea mays

| | MS^{a} | TLC^{a} | |
|---|---|---|---|
| Sterol^{bd} | (M⁺) | (Rf) | Plant Source^{c} |
| Cycloartenol | 426 | 0.29 | st, c, g, r, sh, b, p |
| 24(28)-Methylene-cycloartanol | 440 | 0.29 | st, C, g, r, sh, b, p |
| Cyclosadol | 440 | 0.29 | st, g, sh |
| Cyclolaudenol* | 440 | 0.29 | st |
| Cycloartanol | 428 | 0.29 | sh |
| 24-Methylcycloartanol | 442 | 0.29 | g |
| 24(28)-Methyleneparkeol* | 440 | 0.29 | sh |
| α-Amyrin (triterpene) | 426 | 0.29 | st, c, g, r, sh, b |
| β-Amyrin (triterpene) | 426 | 0.29 | st, c, g, r, sh, b |
| 4α,14α-Dimethylergosta-7,24(28)- | 424 | 0.25 | st, c, g, r, sh, b |
| dienol | | | |
| Lophenol | 400 | 0.25 | g, sh |
| 24-Methylene-lophenol | 412 | 0.25 | c, g, r, sh, b, p, I |
| 24-Methyl-lophenol | 414 | 0.25 | g, sh |
| 24-Ethyl-lophenol | 428 | 0.25 | g |
| Cycloeucalenol | 426 | 0.25 | c, g, r, sh |
| Obtusifoliol | 426 | 0.25 | c, g, r, sh, b, p |
| Dihydroobtusifoliol* | 428 | 0.25 | sh |
| 31-Norlanosterol* | 412 | 0.25 | sh |
| 4α-Methylergosta-8,24(28)-dienol* | 412 | 0.25 | b |
| 4α-Methylergosta-7(E)-23-dienol | 412 | 0.25 | c, g, sh |
| 4α-Methylergosta-7(Z)-23-dienol* | 412 | 0.25 | sh |
| Citrastadienol | 426 | 0.25 | c, g, r, sh, b |
| Isocitrastadienol* | 426 | 0.25 | sh |
| 4α,14α-Dimethyl-ergosta-8(E)-23- | 426 | 0.25 | c, sh |
| dienol | | | |
| 4α,14α-Dimethyl-ergosta-8(Z)-23- | 426 | 0.25 | sh |
| dienol* | | | |
| 4α,14α-Dimethyl-24-ethyl-cholest-8- | 442 | 0.25 | sh |
| enol* | | | |
| 4α,14α-Dimethyl-9,19-cycloergost- | 426 | 0.25 | c, sh |
| 23-enol | | | |
| 4α-Methyl-cholesta- | 410 | 0.25 | sh |
| 8(9),14(1 5),24(28)-trienol* | | | |
| Cholesta-5,22-dienol* | 384 | 0.18 | sh |
| Cholest-7-enol* | 386 | 0.16 | b |
| Cholest-8(9)-enol* | 386 | 0.18 | b |
| Cholesterol | 386 | 0.18 | st, c, g, sh, b, p |
| Cholestanol | 388 | 0.16 | st |
| Brassicasterol | 398 | 0.18 | st, sh |
| 24-Methylene-cholesterol | 398 | 0.18 | st, c, g, sh, b, r, t, p |
| Ergosta-5(E)-23-dienol | 398 | 0.18 | st, c, g, sh, b, r |
| Codisterol | 398 | 0.18 | st, sh |
| Ergosta-7(E)-23-dienol | 398 | 0.16 | st, c, sh |
| 24-Methylene-cholest-7-enol | 398 | 0.16 | st, c, sh, p |
| 24-Methylene-zymosterol | 398 | 0.18 | p |
| Campesterol | 400 | 0.18 | st, c, g, sh, b, r, t, p |
| 24-Epicampesterol | 400 | 0.18 | st, c, g, sh, b, r, p |
| Ergost-(E)-23-enol** | 400 | 0.16 | sh |
| 14α-Methyl-cholest-7-enol* | 400 | 0.16 | sh |
| Ergost-7-enol | 400 | 0.16 | st, c |
| Ergost-8(9)-enol* | 400 | 0.18 | sh |
| Ergostanol | 402 | 0.16 | st, c, sh |
| 24β-Ethylcholesta-5,22,25-trienol | 410 | 0.18 | sh |
| 14α-Methylergosta-8,25-dienol* | 412 | 0.18 | sh |
| 14α-Methylergosta-8,24(28)-dienol* | 412 | 0.18 | sh |
| Stigmasta-7,25-dienol | 412 | 0.16 | sh |
| Stigmasta-8,25-dienol* | 412 | 0.18 | sh |
| 24β-ethyl-cholesta-5,25-dienol | 412 | 0.18 | st, sh |
| Stigmasta-5,23-dienol | 412 | 0.18 | sh |
| Fucosterol | 412 | 0.18 | st, g, sh |
| Isofucosterol | 412 | 0.18 | st, c, g, sh, b, r, t, p |
| 24-Ethylcholesta-5,24(25)-dienol | 412 | 0.18 | st, sh |
| Avensterol | 412 | 0.16 | st, c, sh |
| 25-Methyl-24-methylene-cholesterol* | 412 | 0.18 | sh |
| Stigmasterol | 412 | 0.18 | st, c, g, sh, b, r, t, p |
| Stigmast-7-enol | 412 | 0.16 | c |
| Stigmast-22-enol | 414 | 0.16 | st, sh |
| 14α-methylergost-8(9)-enol | 414 | 0.18 | sh |
| Sitosterol | 414 | 0.18 | st, c, g, sh, b, r, t, p |
| Stigmastanol | 416 | 0.16 | st, sh |

^{a}MS, mass spectrometry; TLC, think-layer chromatography
^{b}*new corn sterol; **new natural sterol
^{c}st, shoot; c, cloeoptile; g, germ oil; sh, sheath; b, blade; r, root; i, inflorescence; t, tassel; p, pollen
^{d}Either trivial or systematic name is given

Biosynthesis of the sterols was analyzed to determine sterol precursor-product relationships. Developmental regulation of sterol metabolism was examined by comparison of different corn tissues. The results show sterols in blades contain mainly 24-ethyl sterols, e.g., sitosterol, while sheaths contained mainly 24-methyl sterols, e.g., 24-methyl-cholesta-5,23-dienol.

Feeding-trapping experiments with four [3-³H]24-methyl sterol isomers incubated with 8-day etiolated sheath tissues indicated that Δ²⁴⁽²⁸⁾-methylene and Δ²⁴⁽²⁵⁾-24-methyl sterols were precursors of 24α- and 24β-methyl sterols, whereas Δ²³⁽²⁴⁾-24-methyl and Δ²⁵⁽²⁷⁾-methyl sterols were end products of the sterol pathway.

The results showed that a single SMT_{I} enzyme is responsible for the catalysis of two methylation steps and that a critical slow step between cycloartenol (start of pathway) and Δ⁵-24-alkyl phytosterol (end of pathway) production is the methylation step, which is subject to feed back regulation from 24-ethyl sterols. The SMT_{I} enzyme regulates the type and amount of phytosterols produced from cycloartenol during plant growth and maturation. This finding contradicts the generally accepted view of the role of 3-hydroxy-3-methylglutaryl-coenzyme A reductase (HMGR). This enzymatic step occurs very early in the isoprenoid pathway from which sterols are derived and has been considered as the rate-limiting step in phytosterol biosynthesis. The present finding shows that HMGR's role is limited merely to controlling carbon flow into the sterol pathway.

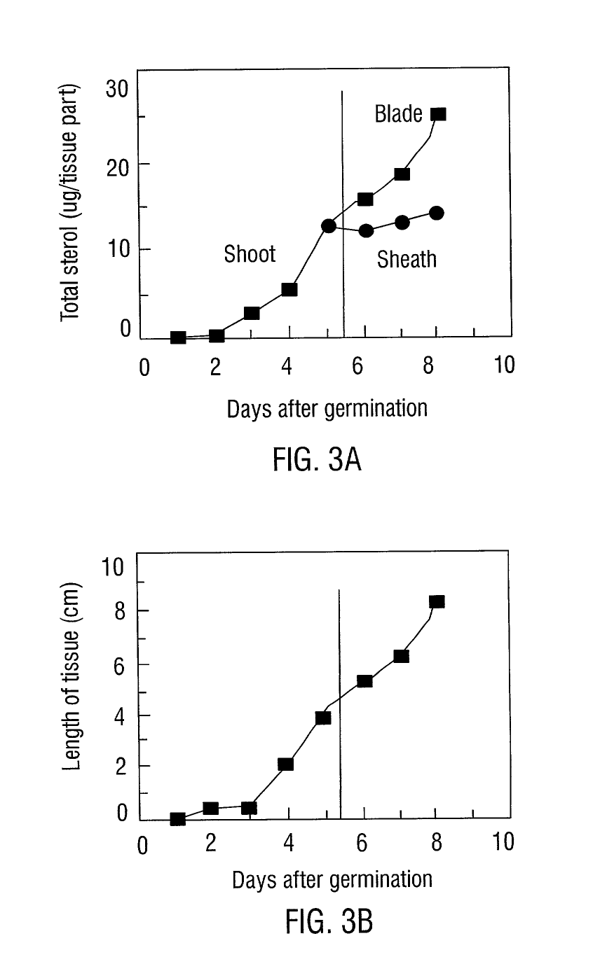

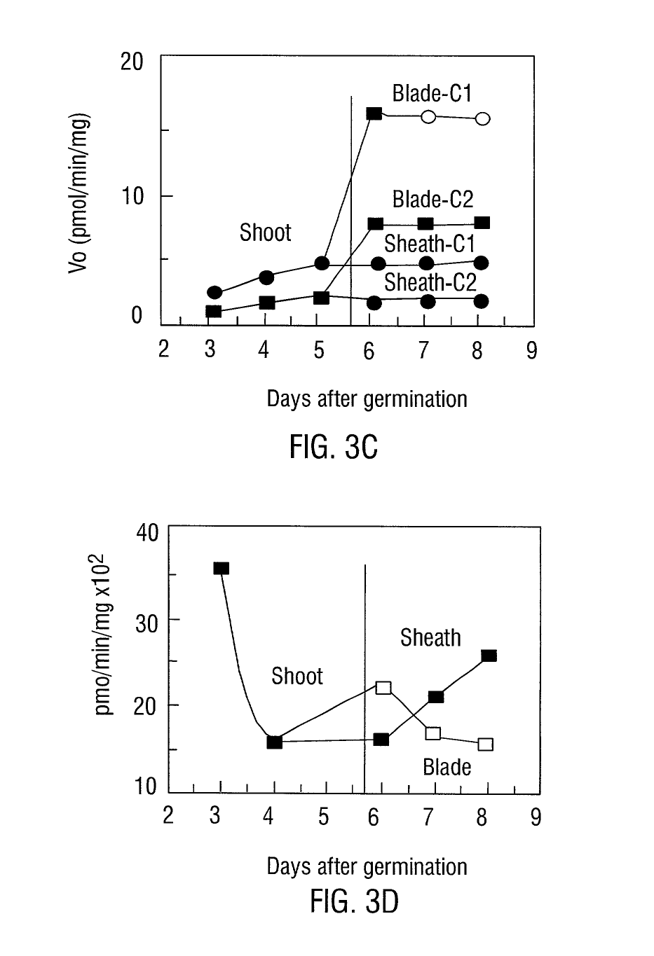

Expression studies of microsomal HMGR activity and microsomal SMT enzyme activity during seedling development following seed imbibition (FIGS. 3C and 3D) show: (1) that SMT activity is correlated with sterol synthesis and plant growth; (2) neither sitosterol nor 24(28)-methylene cycloartanol at 100 mM affected HMGR activity, suggesting that HMGR activity does not correlate to growth or sterol production; and (3) the rate of phytosterol turnover correlates to the activities of the first and second methylation of SMT_{I} enzyme and not HMGR activity.

These results demonstrate that during the initial shoot development following seed imbibition sterol biosynthesis is down-regulated. Sterol that accumulates in 3-day shoots is derived from translocation of sterol originating in the seed. Subsequent corn seedling development results in an up-regulation of phytosterol synthesis. Carbon flow is directed into the phytosterol pathway: Δ⁵-24-alkylsterols are synthesized at rates to meet the increasing demands of membrane synthesis. Cycloartenol and related C-4 methylated sterols are turned over to Δ⁵-end products. The critical slow step, which is the first transformation step in phytosterol synthesis, is methylation of cycloartenol.

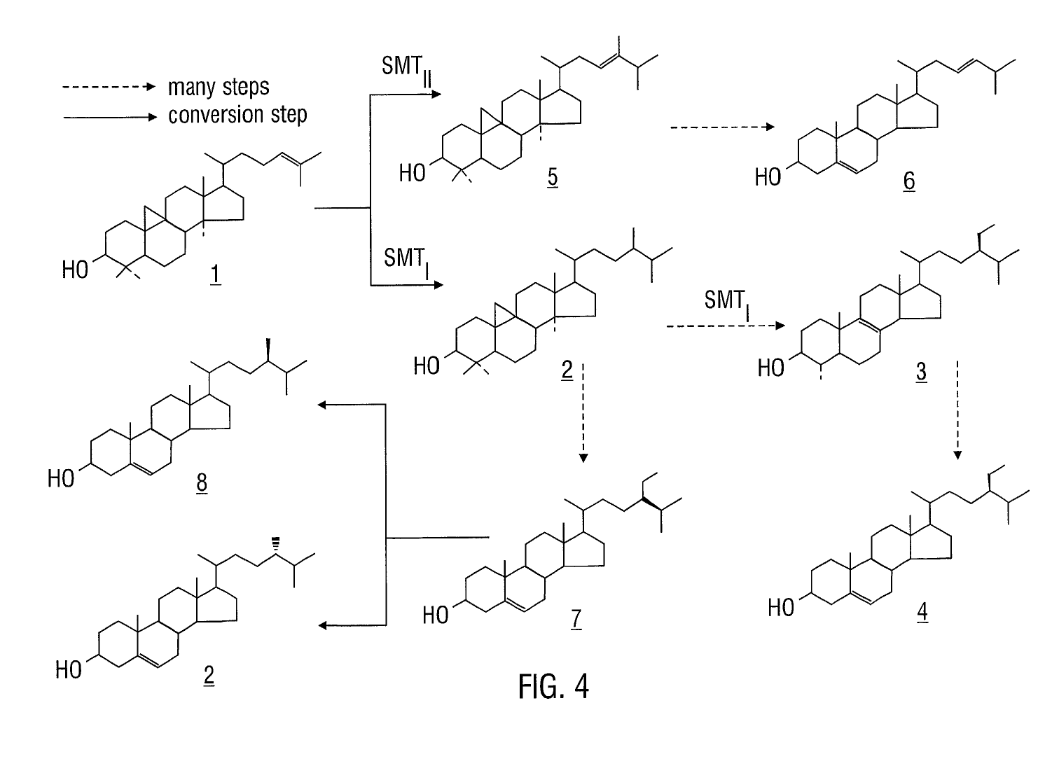

FIG. 4 summarizes the pathway to kinetically favored Δ⁵-24-alkyl sterol end products in corn during development of the seedling into blades and sheaths under dark-grown conditions. Expression of SMT enzyme activities during early blade and sheath formation, and sterol specificity data, show that corn synthesizes at least two different SMT enzymes: SMT_{I} catalyzes the successive methyl transfer to produce Δ²⁴⁽²⁸⁾-methylene and Δ²⁴⁽²⁸⁾-ethylidene sterols; and SMT_{II} catalyzes the methyl transfer to Δ²³⁽²⁴⁾-24-methyl sterols.

### Example 2

### Identification of Sterols Required for Growth of Plants

The phytosterols identified in Example 1 were tested individually for their ability to support growth. In the absence of a plant sterol mutant for such studies the yeast sterol auxotroph, GL-7, was cultured in the presence of sterols identified according to Example 1, above (Li, 1996). This yeast mutant is used as a model system because it can take up sterols from the culture medium and incorporate the test sterol into the membrane lipid bilayer and proliferate. The amount of proliferation of the cells was measured in the presence and absence of hormonal levels of ergosterol, the major yeast sterol.

Sterols were classified according to their effect on growth. Those sterols sparking growth included ergosterol. Those sterols that migrated to membrane and cell structural components without affecting the rate of growth of the cells included cholesterol and sitosterol (Nes et al., 1993).

### Example 3

### Enzymology of Sterol-converting Enzymes

The sterol specificity of the microsome-bound and soluble SMT enzyme from 4-day corn seedlings was determined in order to elucidate the enzymatic basis for the plant sterols identified in Example 1. Using a microsome-bound enzyme system, we observed that cycloartenol is the preferred sterol acceptor and that 24(28)-methylene lophenol was methylated to produce 24(28)-ethylidenelophenol. Table 2 summarizes the specificities to various sterol substrates using the soluble SMT enzyme from corn seedlings.

TABLE 2
Sterol specificity of the
(S)-adenosyl-L-methionine:D²⁴-sterol methyl transferase

| | | % Activity, |
|---|---|---|
| | | relative |
| | Enzyme Activity | to cycloartenol |
| Substrate | (dpm/min) | methylation |
| Cycloartenol | 37,515 | 100 (C1) |
| Lanosterol | 24,384 | 65 (C1) |
| Parkeol | 6,002 | 16 (C1) |
| 31-Norcycloartenol | 18,757 | 50 (C1) |
| 24-Dehydropollinstanol | 8,253 | 22 (C1) |
| Zymosterol | 5,252 | 14 (C1) |
| 4α-Methylzymosterol | 10,504 | 28 (C1) |
| 14α-Methylzymosterol | 3,376 | 9 (C1) |
| 3-Desoxyzymosterol | BG | 0 (C1) |
| Cholest-8-enol | BG | 0 (C1) |
| 24(28)-Methylenelophenol | 3,800 | 10 (C2) |
| 4α-Methylergosta-8,24(28)-dienol | 1,500 | 4 (C2) |
| Obtusifoliol | BG | 0 (C2) |
| Cycloeucalenol | BG | 0 (C2) |
| Ergosta-8,24(28)-dienol | BG | 0 (C2) |
| Ergosta-7,24(28)-dienol | BG | 0 (C2) |
| Ergosta-5,24(28)-dienol | BG | 0 (C2) |
| 24(28)-Methylene cycloartanol | BG | 0 (C2) |

There was little difference in the relative binding efficiencies (Km) of sterols in the microsome-bound and soluble enzyme systems studied. There was a difference in the apparent Vₘₐₓ for the substrates, but this was expected as the level of protein and total sterol endogenous sterol changes during enzyme solubilization. The properties of the soluble SMT enzyme from 4-day corn was similar to that of the microsome-bound SMT enzyme from sunflowers.

The first methyl transfer was demonstrated using cycloartenol and [methyl-³H]-AdoMet incubated with a soluble enzyme preparation from 4-day shoots. In a study on methylation mechanisms operating in corn, [27¹³C]-lanosterol was used to confirm the methylation mechanism producing a 24(28)-methylene sterol in 4-day shoots (Guo et al., 1996).

In neither incubation with cycloartenol or lanosterol was the sterol acceptor molecule methylated to the second methyl product (Nes et al., 1991; Venkatramesh et al, 1996).

The corn SMT protein is an apparent tetramer with 4 subunits of 39 kDa. A bifunctional sterol-methylating (SMT) enzyme was partially purified from 4-day etiolated Zea mays (corn) shoots by the following steps:

(i) non-ionic detergent solubilization of the microsome-bound SMT enzyme;

(ii) gel-filtration fractionation of the solubilized protein to produce active fractions with an apparent native molecular weight of circa 156 kd; and

(iii) hydroxyapatite chromatography of active fractions.

Both methylation activities copurified approximately 200-fold.

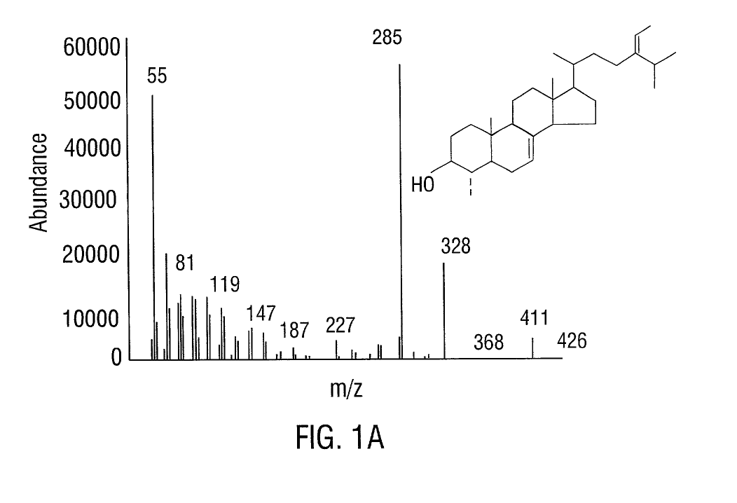

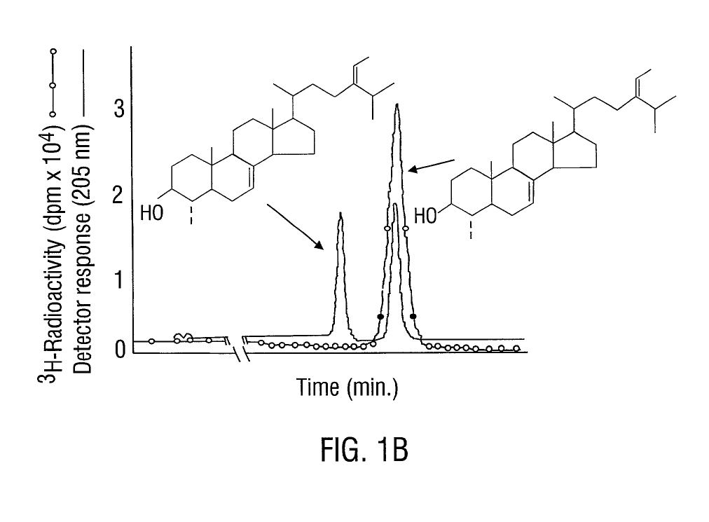

FIG. 1 shows an HPLC-radiocount (FIG. 1B) and mass spectrum (FIG. 1A) of the reaction product from 50 pooled assays from a soluble SMT enzyme (4-day seedlings) assayed with 24(28)-methylene lophenol. The second methyl transfer from 24(28)-methylene lophenol to 24(28)-ethylidene lophenol is demonstrated in this incubation. Thus the SMT enzyme from 4-day corn shoots catalyzes the successive first and second methyl transfers of an appropriate sterol acceptor molecule.

Table 3 shows the effect of a series of substrate and transition state analogs on the first and second methyl transfer reactions.

TABLE 3
Effect of substrate and transition state analog inhibitors on
(S)-adenosyl-L-methionine: Δ²⁴-sterol methyl transferase activity.

| | | | Kᵢ relative to the |
|---|---|---|---|
| | Entry | Kᵢ relative to the | second |
| Inhibitor | no.* | first methyl transfer | methyl transfer |
| Campesterol | 1 | NA | NA |
| 24(28)-Methylene | 2 | 20 μM | NA |
| cycloartanol | | | |
| 26,27-Cyclopropylidene | 3 | 25 μM | NA |
| cycloartenol | | | |
| 24-(R,S)-25-Epimino- | 4 | 55 μM | 55 μM |
| lanosterol | | | |
| Z-24(28)-Ethylidene | 5 | NA | 75 μM |
| lophenol | | | |
| Sitosterol | 6 | NA | 100 μM |

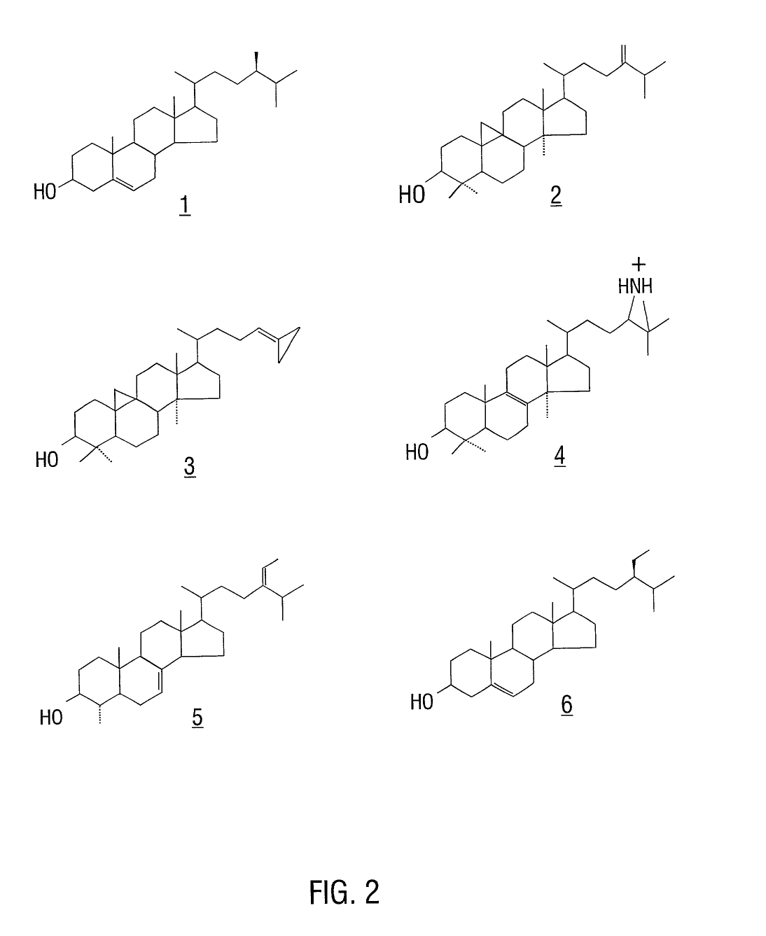

*Numbers indicate structures in FIG. 2

Various inhibitors were tested with soluble SMT enzyme from 4-day seedlings (FIG. 2). The inability of some inhibitors to affect the methylation activity of both sterol substrates suggested that the SMT enzyme has two binding sites.

SMT catalyzes two successive transmethylations from the coenzyme (S)-adenosyl-L-methionine to different substrates: cycloartenol (Δ²⁴-4,4-dimethyl sterol) with 20 mM Km and 4 pmol/min/mg protein Vmax; and 24(28)-methylene lophenol (Δ⁷,24(28)-4-monomethyl sterol) with 11 μM Km and 1 pmol/min/mg protein Vmax. Accordingly, cycloartenol was the preferred substrate for the first methylation reaction and 24(28)-methylene lophenol was the preferred sterol substrate for the second methylation reaction. Zymosterol (Δ^{8.24}-4-desmethyl sterol), a preferred sterol substrate of yeast SMT enzyme, was a poor sterol substrate of the first methylation reaction.

Substrate specificity and inhibition studies suggested substrate binding and release kinetics regulates the first methyl transfer to produce a 24(28)-methylene sterol; and the second methyl transfer to produce a 24(28)-ethylidene sterol.

For Example, sitosterol (24α-ethyl cholesterol), the major end product of corn sterol production in blade tissue, inhibited the second methyl transfer (100 PM Kᵢ), without affecting the first methyl transfer; campesterol (24α-methyl cholesterol) failed to inhibit either the first or second methylation reaction; 24(28)-methylenecycloartanol, a product of cycloartenol transmethylation, was not methylated; and 24(28)-methylenecycloartanol inhibited the first methyl transfer (20 μM Kᵢ) whereas it failed to inhibit the second methyl transfer. 26,27-Cyclopropylidene cycloartenol, which failed to bind to the yeast SMT enzyme, was a potent competitive inhibitor of the first methylation reaction (25 μM Kᵢ), while not affecting the second methyl transfer.

The second alkylation was inhibited by product inhibition from 24(28)-ethylidene lophenol (75 mM Kᵢ), while not affecting the first methyl transfer. A transition state analog, 24-(R,S)-25-epiminolanosterol inhibited the first and second methylation reactions with a similar Kᵢ value of 55 nM and to exhibit a non-competitive type kinetic pattern. The sterol features of the substrate in the initial enzyme-substrate interaction appears to be typical of other plant SMT enzymes, i.e., a requirement for nucleophilic groups at C-3 and C-24. The 5 μM Kₘ for the coenzyme was the same for the first and second methylation reactions.

### Example 4

### SMT Genes from Yeast

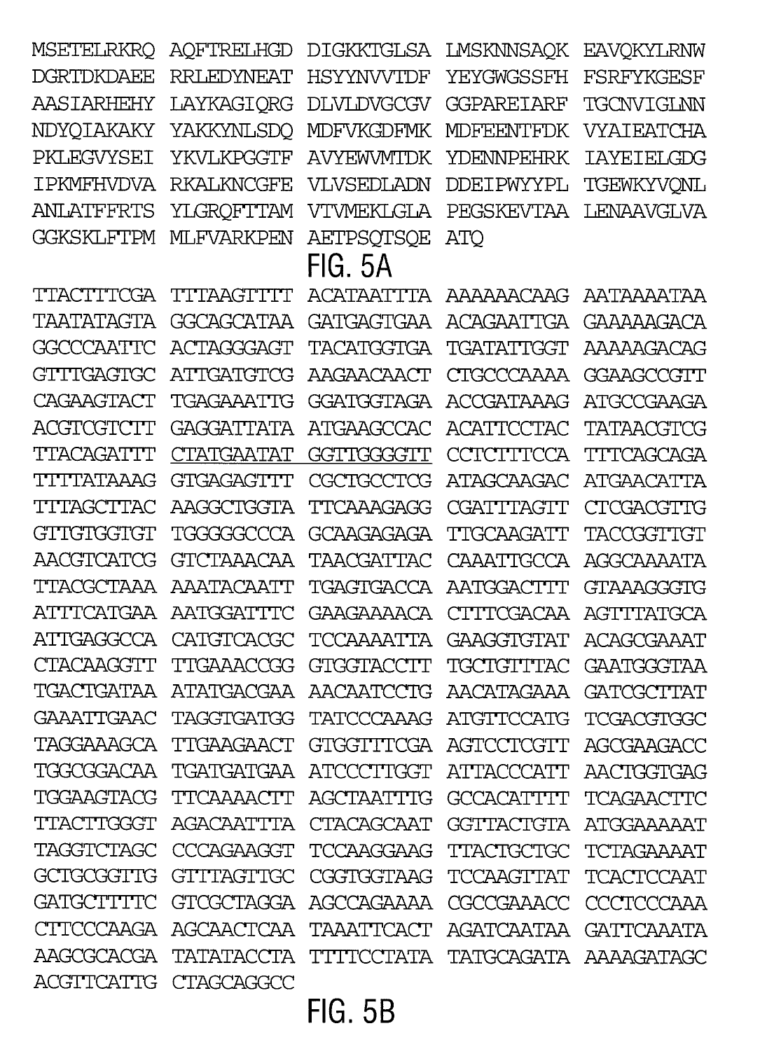

The yeast SMT gene, ERG6, was derived from a yeast ERG6 genomic fragment, pRG458/erg6 (FIG. 5B; SEQ ID NO:1).

The cloned ERG6 gene was expressed in E. coli (Venkatramesh et al., 1996). The recombinant protein was shown to be the sterol biomethylation enzyme by enzymatic study which proved that the kinetic properties were similar to that of the native enzyme in yeast. In contrast to plant SMT which prefer cycloartenol, zymosterol, a Δ²⁴-4-desmethyl sterol, is the preferred substrate of the yeast SMT.

The molecular weight of the yeast SMT monomer was confirmed to be 43 kD after successfully overexpressing the active protein in E. coli using a T7 promoter-based pET23a(+) vector. The overexpressed protein was visualized on SDS-PAGE gel both by Coomassie blue staining and Western blot using a yeast SMT polyclonal antibody. The recombinant protein has also been purified from this system (Nes et al., 1998).

From the deduced amino acid sequence of the yeast SMT (FIG. 5A; SEQ ID NO:2) the potential sterol binding motif was predicted as the first conserved region identified in FIG. 5A (YEYGWGS) and based on mechanistic analysis of biomethylation described in Example 3, the amino acid tryptophan (W) was determined to be associated with the putative sterol binding site. By site-directed mutagenesis of the ERG6 gene this amino acid was replaced with alanine. The mutated DNA was also overexpressed in E. coli by cloning into pET23a(+). This protein was not active under conditions where the wild-type protein was active.

Such a strategy provides a means to alter phytosterols by introducing inactive SMT protein into plants. The introduction of non-functional SMT monomers can result in the suppression of SMT activity, for example by affecting the ability of the cell to form a functional SMT enzyme complex, thereby leading to the formation of nonutilizable sterols. For example, suppressing the activity of the first SMT_{I} reaction will lead to formation of Δ²³⁽²⁴⁾-24-alkyl sterols, products of SMT_{II} activity. Alternatively, suppressing the activity of the second SMT_{I} reaction will lead to the formation of Δ²⁴⁽²⁵⁾-24-alkyl sterols.

### Example 5

### SMT Genes from Arabidopsis

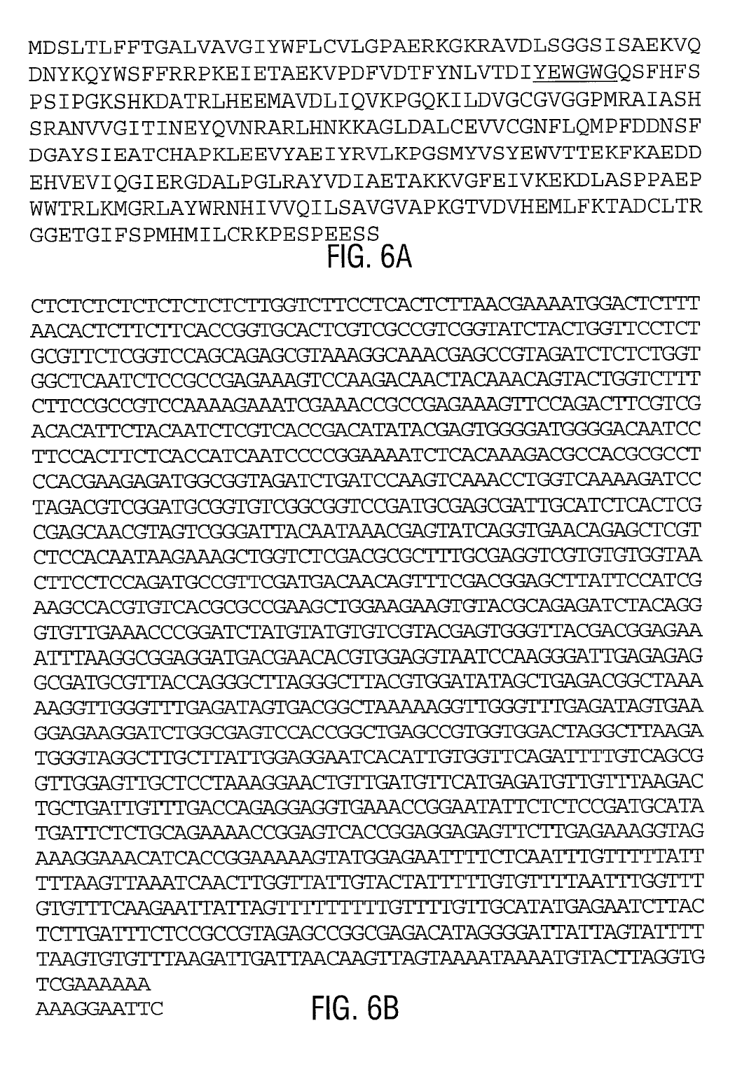

The SMT gene from Arabidopsis was cloned and sequenced (FIG. 6; SEQ ID NO:3). This gene containing a His-tag was overexpressed in E. coli (Tong et al., 1997). Arabidopsis SMT was partially purified and characterized in stereochemical detail.

The Arabidopsis SMT gene was amplified by PCR from a cDNA library. The primers used were designed from the full-length cDNA sequence retrieved from the GeneBank (Accession number X89867). The amplified product was the full-length Arabidopsis SMT gene which was sub-cloned into a T/A cloning vector and sequenced. From the sequence data the ORF was identified. A Nde I site was created at the ATG start codon through PCR mediated site-directed mutagenesis. The full-length ORF containing a Nde 1 site at the start and a BamH I site at the stop was cloned into the pET23a(+) vector just as the ERG6 gene was in Example 4. The recombinant fusion protein was active in transforming both cycloartenol and 24(28)-methylene lophenol to their respective alkyl products. In the case of cycloartenol only one product was formed, which is 24(28)-methylene cycloartanol, i.e., SMT_{I} in FIG. 4. Since a single gene product was able to metabolize both sterol substrates it further confirms the enzymological data in Example 3. Further, since cycloartenol metabolism by the recombinant plant SMT gave rise to only one product which also is the product of SMT_{I} it suggests that the alternate product, cyclosadol (structure 6 in FIG. 4), is formed from a different protein (SMT_{II}) encoded by a unique sterol gene.

### Example 6

### SMT Genes from Corn

The corn sterol methyl transferase (SMT) gene was isolated from a commercial corn cDNA library (Stratagene, La Jolla, Calif.). Five microliters of corn cDNA (equivalent to 5×10⁷ pfu) were used as template in the amplification of the SMT gene by polymerase chain reaction (PCR). Because the cDNA library was constructed in the vector Uni-Zap XR (Stratagene), the T7 sequence in this vector was used as one of the two primers for PCR amplification (3 ′end primer). The 5′ end primer (2650-1) was designed from nucleotides 2-20 of a putative SMT fragment published in Gene Bank (T23297). Thirty cycles of PCR were conducted using five units of Taq polymerase from Promega in a total volume of 100 microliters, according to the manufacturer's instructions. One microliter of PCR product from this reaction was used as the template for a second round of PCR using the T7 primer and a primer designed from nucleotides 250-268 of T23297. When the resulting reaction products were analyzed on a 1% agarose gel, a band of 1.3 kb was seen. This PCR band was subcloned into the plasmid pGEM-T (Promega) and was sequenced.

To obtain the 5′ end of the SMT gene, a pair of primers designed from nucleotides 2-20 and 366-349 of sequence T23297, was used in the PCR amplification. A band of 366 nucleotides was obtained and sequenced. The sequence of this 366-nucleotide PCR fragment overlapped with the 1.3 kb clone for 116 nucleotides. These two fragments were joined together by PCR, using a pair of primers, 2650-1 and 3082-2. The latter primer was designed from the 1.3 kb fragment 20 nucleotides before poly A sequence. Both of the 366 bp and the 1.3 kb PCR fragments were used as the DNA templates. The reconstructed SMT gene was ligated to the PCR cloning vector pGEM-T and was sequenced bi-directionally using the ABI Prism Automatic DNA Sequencer (Model 310).

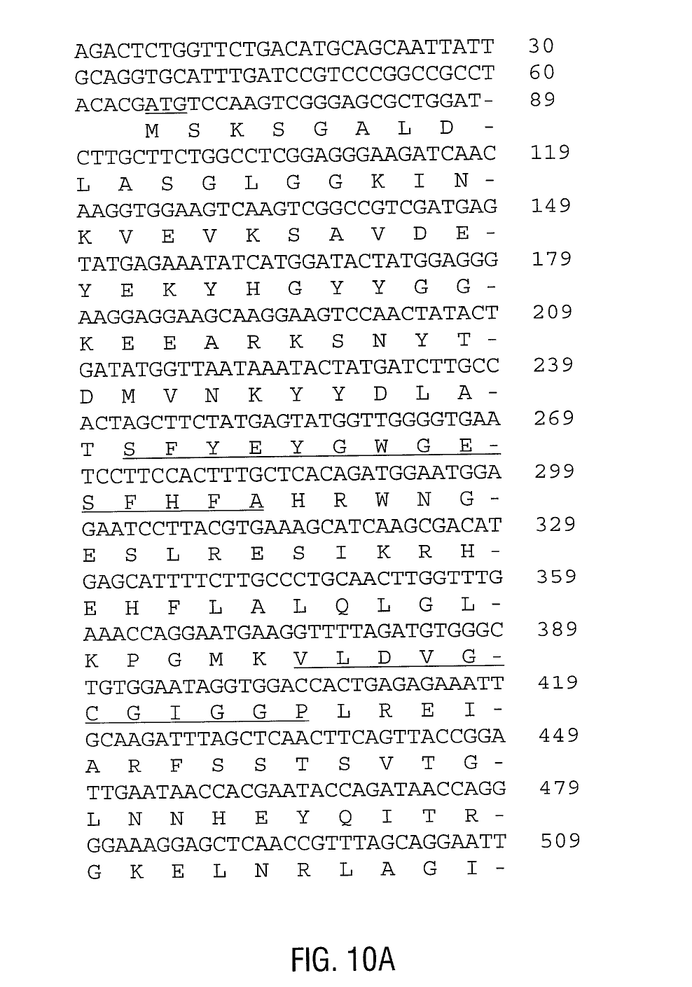

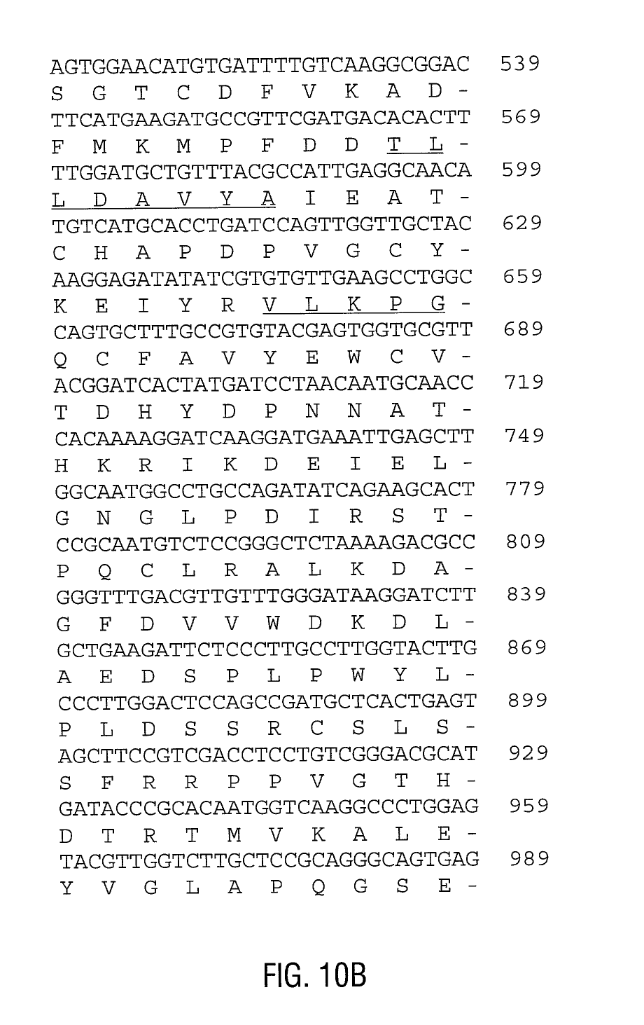

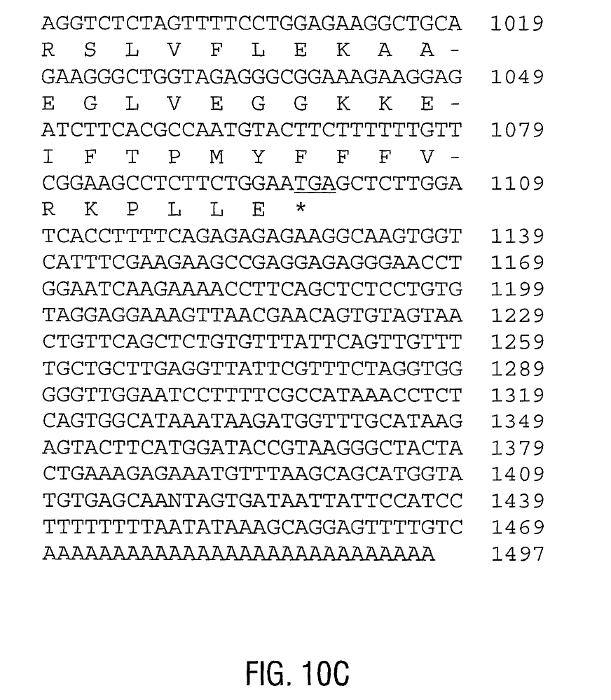

The cloned SMT cDNA was 1497 nucleotides, with a coding region of 1032 nucleotides, which encodes 344 amino acids (FIG. 10; SEQ ID NO:6). The start codon, ATG, was located at nucleotide 66-68. There was one stop codon preceding the start codon (ATG), located at position 42-44, suggesting that the reconstructed SMT sequence contains the complete 5′end. A poly A tail of 28 nucleotides was located 371 nucleotides downstream of the stop codon, indicating the cDNA fragment was complete at 3′end. Therefore, this cDNA clone is a full length cDNA clone.

The deduced amino acid sequence from this cDNA clone contains 344 amino acids, encoding a polypeptide of 38.8 kiloDaltons. This deduced amino acid sequence contains all three of the proposed conservative regions for methyl transferase (Kagan and Clarke, 1994. Arch. Biochem. Biophys. 310: 417-427): LDVGCGIGGP at position 104-114 (amino acid sequence) and TLLDAVYA at position 167-174, and VLKPGQ at position 194-199. In addition, another conserved region tentatively assigned for the sterol binding site, proposed by Nes (SFYEYGWGESFHFA; Guo et al.,1997). Antifungal sterol biosynthesis inhibitors. In Subcellular Biochemistry Volume 28: Cholesterol: Its function and Metabolism in Biology and Medicine, edited by Robert Bittman. Plenum Press, New York), was observed at position 60-73.

The deduced corn SMT amino acids sequence was compared with amino acid sequences from other known SMT genes using GCG progams (Gap and Bestfit). The deduced corn SMT amino acid sequence shared a 93.6% similarity with an independently isolated corn SMT sequence (Genbank U79669), 88.1% homology, 78.8% identity with soybean SMT (Genbank U43683), and a 93.9% homology, 88.3% identity with partial wheat SMT sequence (Genbank U60754), 58.8% homology, 39% identity with Arabidopsis thaliana (Genbank X89867), and a 66.5% homology, 50.4% identity with yeast SMT (Genbank X74249). The high similarity between this cDNA clone and SMT genes from other plant species confirms that this cDNA clone is a full length SMT cDNA clone of Zea mays. Furthermore, since Grabenok et al. have functionally expressed their corn SMT gene in a yeast expression system and found no 24-alkyl sterols other than ergosterol, this suggests that the corn SMT gene isolated by my laboratory catalyzes the same stereoselective C-methylation to Δ²⁴⁽²⁸⁾, thereby supporting the view that corn synthesizes several different SMT enzymes.

A similar strategy can be used for isolating the cDNA for the SMT_{II} gene. In fact, cDNA fragments isolated by the described method should be representative of both SMT_{I} and SMT_{II} based on the conservation of the region from which the primers were derived.

### Example 7

### SMT Genes from Prototheca wickerhamii

Another example of a preferred SMT gene is that from Prototheca wickerhamii. This yeast-like alga produces Δ²⁵⁽²⁷⁾-24-methyl sterol as the main product of transmethylation (Nes et al., 1990). The favored substrate is cycloartenol.

Studies from microsome preparations of P. wickerhamii have shown that the preferred substrate of the SMT is cycloartenol. However, the preferred product is not 24(28)-methylene cycloartenol but cyclolaudenol (VII) which is a Δ²⁵⁽²⁷⁾-24-alkyl sterol, a nonutilizable sterol.

Cloning the gene of this SMT will facilitate the introduction of this gene into plants in order to transform the plant sterol, cycloartenol, into a product, cyclolaudenol, which will lead to the accumulation of nonutilizable sterols, viz., Δ²⁵⁽²⁷⁾-24-alkyl sterols.

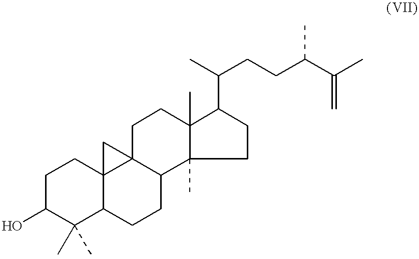

Cloning of Prototheca SMT

Prototheca wickerhamii cells are grown to mid log phase in YPD rich medium (yeast extract—peptone—dextrose). The pelleted cells are disrupted in the presence of Tri Reagent (MRC) using 0.5 mm glass beads and a mini-Beadbeater (both from Biospec Products, Bartlesville, OK). High quality total cellular RNA is isolated according to the manufacturer's instructions. Heterologous expression of the resulting sterol gene is used to confirm the identity of the cloned gene by assaying the expressed protein for characterization and mechanistic studies.

Using a homology based PCR strategy, total cellular RNA is subjected to 3′ RACE (rapid amplification of cDNA ends) and 5′ RACE using reagents and protocols found in kits obtained from GibcoBRL. For 3′ RACE, total cDNA is synthesized by the action of reverse transcriptase after annealing oligo(dT)-containing primers to the poly(A)-tailed RNAs present in the unfractionated total RNA. The RNA templates are degraded and the cDNA serves as template for polymerase chain reaction (PCR) amplification. The user-supplied primer “YEYGWG” (see Rationale for primer design below) anneals to the cDNA and is extended toward the 3′ end of the gene under the direction of Taq polymerase. The kit-supplied primer for extension from the 3′ end to the terminus defined by the “YEYGWG” primer anneals to a sequence composed of three restriction endonuclease recognition sites that was part of the original oligo-dT containing primer. A second PCR amplification in which the primer pair is a second “nested” primer (“GCGVC-G”) and the kit-supplied 3′ primer is performed to enrich for cDNAs representing the 3′ half of SMT. Another nested primer (“ATCHAP”) has been similarly used.

Total cellular RNA is also subjected to 5′ RACE. cDNA is synthesized by reverse transcriptase using the antisense primer “EWVMTDas”. cDNA is modified at the 3′ end by the addition of a polydeoxycytidine “tail” using terminal deoxynucleotidyl transferase (TdT). An initial PCR reaction is carried out using this C-tailed CDNA as template and the primers “EWVMTDas” and a kit-supplied poly-G containing primer. A second PCR reaction is carried out on this PCR product using the nested primer “ATCHAPas” and a kit-supplied primer that anneals to a part of the poly-G primer that contains restriction enzyme recognition sites. This second PCR reaction enriches for 5′ SMT cDNA sequences.

The 3′ RACE and 5′ RACE PCR products are isolated from gels and ligated into the plasmid pPCRII (Invitrogen). Clones obtained after transformation into E. coli are characterized by sequencing. An Apa I restriction site is present in the DNA of all plants and yeast that have been sequenced in the GCGVGG motif and is present in both the 3′ and 5′ cDNA clones. This allows splicing of the two 3′ and 5′ halves of the SMT gene together, to give rise to a full length SMT gene.

Primers designed from internal conserved regions and primers designed from both 5′ and 3′ ends of the first strand cDNA are used to amplify the SMT gene from the 5′ and 3′ end, respectively. Different annealing temperatures (35° to 60°) and different amounts of cDNA templates are tested to amplify the SMT gene.

The coding of this SMT gene is subcloned into expression vector pET23 for protein expression and enzymatic assay (Nes et al., 1998).

Rationale for Primer Design

Comparison of the six available SMT amino acid sequences allows for definition of four conserved regions (Nes et al., 1998). The first step in designing the user-supplied primers was to examine the several very highly conserved peptide motifs in the SMTs of those plants and yeast that have been sequenced. Within these are found shorter stretches of amino acid sequences that can be encoded by a minimum number of DNA sequences, the codons of which usually only vary at the third (degenerate) base. It was also desirable that the codon preferred by 3 different yeast species according to codon usage tables found in Wada, et. al. (Nucleic Acids Res., vol 19, p1981, 1991) be present in the mix of degenerate codons for each amino acid. Each user defined primer is thus a mixture of deoxynucleotides that defines an internal end of a PCR product. It was also reqiured that 4 or 5 of the 6 3′ deoxynucleotides of each primer be perfectly matched in all species and had greater than 50% G and/or C.

The first three primers described below are sense orientation primers that anneal to antisense DNA (and the original cDNA). The fourth and fifth primers are antisense primers that anneal to the sense DNA strand of the SMT gene.

YE[Y/F/W]GWG (amino acids 81-86 of the yeast sequence; nonidentical residues at a position are in brackets) was the part of the conserved region of SMT enzyme that was the basis for the “YEYGWG” primer and is considered the sterol binding site:

5′-TA[T/C]GA[A/G]T[A/G/T][T/G]GG[T/A/C]TGGGG-3′

(Degenerate nucleotide positions are included in brackets)

The “GCGVGG” primer was suggested by the DNA sequence that encodes part of a second conserved domain (GCG[V/I]GG) at yeast amino acid residues 129-134. The sequence of primer “GCGVGG” is:

5′-GGATG[T/C]GG[T/A][G/A]T[T/C]GG[G/C]GG-3′.

Primer “ATCHAP” is based on the DNA sequence encoding a third highly conserved domain (yeast amino acids 196-203). The primer sequence is:

5′- GCCAC[A/G/T]TG[T/C]CA[C/T]GC[T/G/A]CC-3′.

Primer “EWVMTDas” is an antisense primer for first strand cDNA synthesis in the 5′ RACE experiment. It is based on the small conserved domain at yeast amino acid residues 225-231. The sequence is:

5′-TC[A/C/G]GTC[G/A]T[T/A/G][C/A][C/A]CCA[C/T]TC-
3′.

Primer “ATCHAPas” is a nested antisense primer for the 5′ RACE experiment with the sequence:

5′-GG[T/C/A]GC[A/G]TG[G/A]CA[A/C/T]GTGGC-3′.

### Example 8

### SMT Genes from Other Plants

Using the Arabidopsis cDNA or another plant derived SMT sequence as a probe, CDNA libraries from any crop of interest can be screened and corresponding clones of appropriate sizes can be isolated and sequenced. CDNA library construction and screening methodologies are well known in the art. As described in Example 6, appropriate primer combinations can be readily determined using information of the conserved regions of known sequences for various SMT genes. To confirm the identity of sequences cloned by this method, they can be compared with known plant SMT enzyme sequence and/or in vitro tranlsated and evaluated biochemically.

### Example 9

### Plant Transformation with ERG6 DNA

To obtain transgenic plants with altered sterol profiles a DNA fragment containing the open reading frame of the SMT ERG6 gene of yeast isolated from a genomic clone was identified (Example 4). The ERG6 DNA was modified by PCR to include restriction sites for Nco I on either end of the open reading frame. This PCR procedure gave ruse to a mutaion which introduced a frameshift in the gene. This mutation made the ERG6 gene introduced into the plant untranslatable, but capable of inhibiting the endogenous tomato SMT via antisense or co-suppression mechanisms, depending upon the nature of the construct.

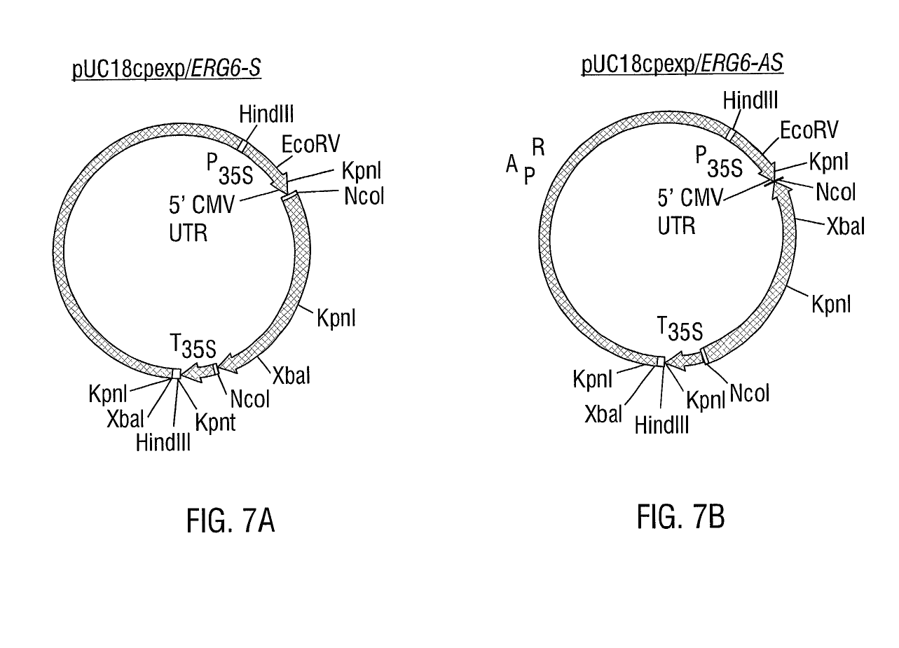

The modified ERG6 DNA fragment was cloned into the pUC18cpexp expression cassette vector. Clones with the ERG6 DNA in the sense as well as the antisense orientations to the 35S promoter were generated (FIG. 7).

Hind III digestion of these clones gave rise to the ERG6 constructs that included the 35S promoter and termination sequences flanking the ERG6 open reading frame. These Hind III digested fragments were cloned to the binary vector pJTS246 that contains T-DNA border recognition sequences and the NPTII gene conferring kanamycin resistance.

The cloned binaries with either the sense or antisense ERG6 constructs were transformed into Agrobacterium tumefaciens which were cocultivated with cotyledons of tomato (Solanum lycoperiscum) to obtain transformed plant cells. From calli formed on selective medium containing kanamycin transgenic plants were produced.

The leaves from control (no inserts) and transgenic plants (with inserts) were analyzed for the transgene. DNA was extracted from leaf samples of each of the transformants and an untransformed tomato plant. The DNA extracts were quantified by A260 absorbance.

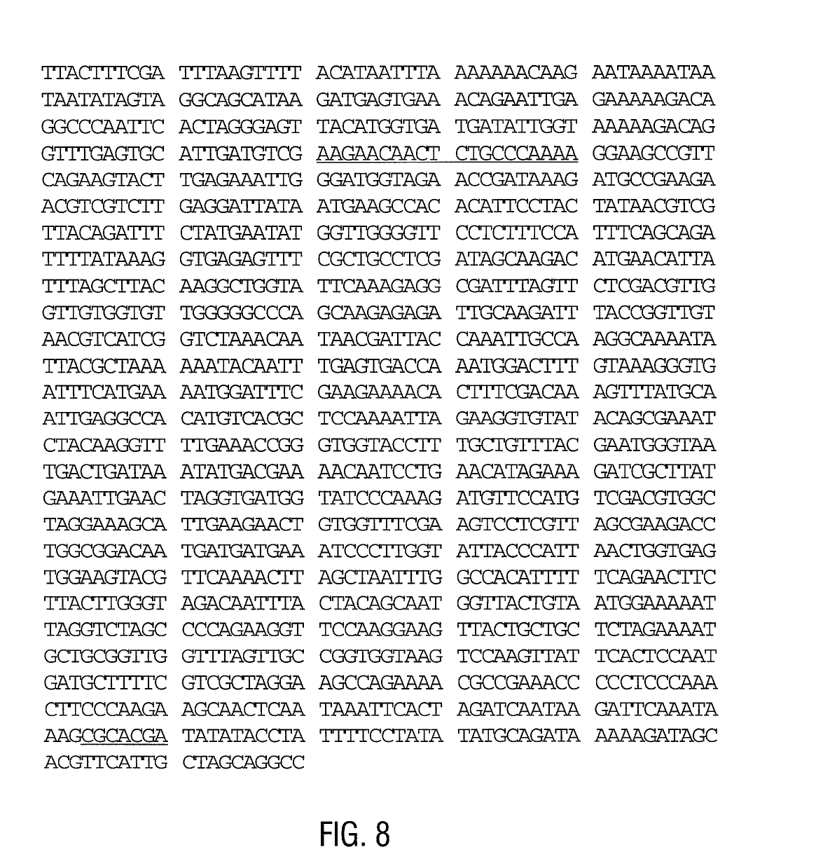

Aliquots corresponding to 200 ng DNA from each sample were used in PCR reactions for amplifying ERG6 fragments using oligonucleotide primers corresponding to the ERG6 sequence (underlined in FIG. 8). Controls in the PCR included a sample with no template DNA and samples of the sense and antisense ERG6 containing binary plasmids. PCR was performed under non-stringent conditions (55° C. annealing temperature for 2 min in each cycle) in 20 cycles and aliquots were electrophoresed on 0.8% agarose gels.

The primers were selected such that a 1100 bp fragment of the ERG6 DNA would be amplified (FIG. 8). All the regenerated transgenic tomato plants (R₀) carried this fragment as did the plasmid controls. There also is some non-specific amplification because of the non-stringent conditions leading to other bands appearing in the transformed plants and in the untransformed control. However, the level of these amplifications is significantly less than that of the target fragment. This confirms the presence of the ERG6 DNA in the tomato genome.

Sterol analysis was performed on the nonsaponifiable lipid fraction of leaf material from one regenerated plant transformed with the sense construct and one regenerated plant transformed with the antisense construct. The results are shown in Table 4.

TABLE 4
Sterol Composition of Tomato Plants
(as % total sterol)

| | | ERG6 sense | ERG6 antisense |
|---|---|---|---|
| Sterol | Control | insert | insert |
| Cholesterol | 29 | 18 | 20 |
| Cholest-7-enol | none | 21 | 13 |
| Stigmasterol | 25 | 22 | 24 |
| Sitosterol | 26 | 27 | 24 |
| Isofucosterol | 20 | 12 | 19 |
| mg sterol/g fr.wt. | 16 | 150 | 380 |

The result confirmed that the ERG6 gene was incorporated into the transgenic plants and that the sterol compositions of the transgenic plants were changed. A novel sterol, cholest-7-enol, which is not present in control tomato plant leaves, was detected and characterized by mass spectroscopy.

A scheme for the new pathway introduced into the tomato plants due to the insertion of the yeast ERG6 gene is predicted to be as follows:

Since both the sense and antisense inserts of the ERG6 gene lead to the accumulation of the cholest-7-enol (VIII), it is likely that in both cases there is a suppression of endogenous SMT activities. This will lead to a shunt of carbon flow into an alternate minor pathway proposed for phytosterol metabolism where the first step in cycloartenol metabolism is a reduction of the C-24 double bond by a reductase enzyme. The resulting sterol, which is cycloartanol (IX), will then undergo the usual demethylation, isomerization, desaturation and reduction just as in the main pathway leading to the formation of cholest-7-enol. This is a Δ⁷-sterol and the double bond at C-5 is absent, suggesting that some insects will not be able to utilize this sterol to complete their life cycles.

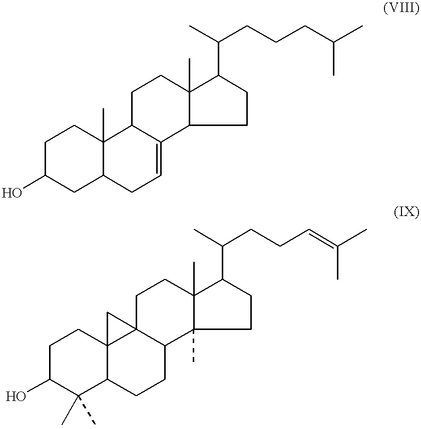

The regenerant (R₀) plants were allowed to flower and set fruit. Seeds were collected, and the following generation (R₁) was grown. Individual plants arising from seeds were assayed for the presence or absence of the selectable marker (NPT2) via BLISA assay for the NPT2 protein. Fifty-three plants from six R₁ progeny and a nontransgenic plant were analyzed for sterol composition. The sterol profiles of these plants could be divided into four distinct groups, or phenotypes:

TABLE 5
Means and standard deviations (Std) of sterols (as percent of total
sterols) of R₁ plants in the four classes of progeny identified.

| Phenotype | 1 | | 2 | | 3 | | 4 | |
|---|---|---|---|---|---|---|---|---|
| Sterol | Mean | Std | Mean | Std | Mean | Std | Mean | Std |
| Cholesterol | 7.62 | 2.54 | 6.20 | 2.77 | 4.93 | 1.14 | 8.60 | 2.97 |
| Campesterol | 4.17 | 3.15 | 16.60 | 11.24 | 4.50 | 1.95 | 6.60 | 4.83 |
| Stigmasterol | 13.14 | 3.13 | 12.80 | 5.26 | 8.86 | 1.41 | 22.60 | 1.14 |
| Sitosterol | 11.48 | 2.86 | 11.60 | 2.19 | 9.57 | 1.87 | 16.60 | 3.91 |
| Isofucosterol | 13.14 | 2.08 | 7.60 | 3.71 | 9.86 | 2.32 | 14.40 | 4.98 |
| b-Amyrin | 12.52 | 3.90 | 9.75 | 5.91 | 10.36 | 3.95 | 8.80 | 1.79 |
| Cycloartenol | 31.76 | 5.67 | 31.60 | 4.72 | 49.36 | 4.91 | 28.80 | 6.98 |
| 24(28)- | 1.14 | 1.46 | 6.80 | 6.61 | 2.17 | 2.12 | 2.00 | 2.00 |
| methylene | | | | | | | | |
| cycloartanol | | | | | | | | |

All of the R₁ plants which tested negative for the NPT2 marker (and were therefore non-transgenic segregants) as well as the nontransgenic control plant displayed the normal phenotype (Phenotype 1). The R₁ plants which tested positive for the NPT2 marker (and were therefore transgenic) fell into all four classes. A statistical comparison was conducted for each sterol (using the arcsin transformation of the percent sterol levels; Student-Neuman-Keuls Test, 5% significance level), and a qualitative summary of the results is given below:

TABLE 6
Comparison of sterol phenotypes
(Phenotypes 2, 3 and 4 versus normal Phenotype 1)

| Sterol | Phenotype 1 | Phenotype 2 | Phenotype 3 | Phenotype 4 |
|---|---|---|---|---|
| Cholesterol | Normal | Normal | Low | Normal |
| Campesterol | Normal | High | Normal | Normal |
| Stigmasterol | Normal | Normal | Low | High |
| Isofucosterol | Normal | Low | Low | Normal |
| β-amyrin | Normal | Normal | Normal | Normal |
| Cycloartenol | Normal | Normal | High | Normal |
| 24(28)- | Normal | Normal | Normal | Normal |
| methylene | | | | |
| cycloartanol | | | | |
| Sitosterol | Normal | Normal | Normal | High |

The distribution of plants in the various categories (i.e. nontransgenic controls in the normal category only and the transgenics plants in all four categories) is consistent with the expectations of plants resulting from transformation with either an antisense or co-suppression construct. Varying levels of suppression can be expected between and within progenies, thus leading to varying levels of expression of an altered sterol phenotype. Therefore, these results are consistent with the transformed ERG6 gene having a suppressive effect. More specifically, phenotypes 2 and 3 accumulate intermediates which are consistent with partial inhibition of the first or second methylation activities of sterol methyltransferase in the biosynthetic pathway. The elevated levels of sitosterol and stigmasterol (the normal endproducts) are not consistent with suppression, and cannot be explained without further study.

Independent analyses of a subset of these progeny further supports the hypothesis that suppression of the SMT gene is being observed in the transgenic lines. Table 7 below gives the sterol compositions of nontransformed and nontransgenic segregants.

TABLE 7
Sterol composition of control plants (nontransformed plants
and nontransgenic segregants)

| | Non- | G55 (non- | G62 (non- | | |
|---|---|---|---|---|---|
| | trans- | transgenic | transgenic | | Std. |
| Plant | formed | segregant) | segregant) | Mean | Dev. |
| Sterol | | | | | |
| Cholesterol | 18 | 13 | 13 | 14.7 | 2.9 |
| Δ⁰-Cholesterol | — | tr. | 1 | 1.0 | |
| 14-α-CH₃-Δ7- | — | 5 | 5 | 5.0 | 0.0 |
| Cholesterol | | | | | |
| Δ7-Cholesterol | — | — | — | | |
| 14-α-CH₃-Δ8- | 3 | 1 | 1 | 1.7 | 1.2 |
| cholesterol | | | | | |
| Zymosterol | 18 | — | — | 18.0 | |
| Δ^{7,24}-Zymosterol | 5 | — | — | 5.0 | |
| 24-CH₂- | — | 19 | 1 | 10.0 | 12.7 |
| Cholesterol | | | | | |
| Campesterol | 2 | 8 | 3 | 4.3 | 3.2 |
| Desmosterol | 2 | — | | 2.0 | |
| Δ⁰-Campesterol | — | — | 1 | 1.0 | |
| Stigmasterol | 18 | 20 | 25 | 21.0 | 3.6 |
| Δ⁰-Stigmasterol | — | tr. | 1 | 1.0 | |
| Sitosterol | 7 | 13 | 18 | 12.7 | 5.5 |
| Δ⁰-Sitosterol | — | — | tr. | | |
| Isofucosterol | 4 | 2 | 2 | 2.7 | 1.2 |
| Cycloartenol | 7 | 19 | 29 | 18.3 | 11.0 |
| 24-CH₂- | 14 | — | tr. | 14.0 | |
| Cycloartenol | | | | | |
| 24-CH₂-Lophenol | 1 | — | tr. | 1.0 | |
| Obtusifoliol | 1 | — | tr. | 1.0 | |

—dash lines means not detected; tr. is trace; N.D. - not determined. NSF was chromatographed on TLC plates and bands matching 4-desmethyl-, 4-monomethyl and 4,4-dimethyl sterol standards were eluted from the plate and examined further by chromatography on 3% SE-30 columns and GC-MS. Limit of detection is 0.1 mg sterol per leaf sample.

These controls can be compared with transgenic plants, the sterol composition of which are given in tables 8, 9, and 10.

TABLE 8
Sterol composition of transgenic plants from line G3

| Plant | G31 | G32 | G34 | G35 | G37 | G38 | G39 |
|---|---|---|---|---|---|---|---|
| Sterol | | | | | | | |
| Cholesterol | 12 | 10 | 8 | 10 | 8 | 11 | 8 |
| Δ⁰-Cholesterol | 1 | tr. | 1 | 1 | 1 | tr. | 1 |
| 14-α-CH₃-Δ⁷- | 3 | — | — | — | — | — | 3 |
| Cholesterol | | | | | | | |
| Δ⁷-Cholesterol | — | 8 | 6 | 13 | 11 | 1 | — |
| 14-α-CH₃-Δ⁸- | 1 | 2 | 2 | — | — | — | — |
| cholesterol | | | | | | | |
| Zymosterol | 10 | 5 | 12 | — | — | — | 8 |
| Δ^{7,24}-Zymosterol | 2 | — | 1 | — | — | — | 1 |
| 24-CH₂-Cholesterol- | — | — | — | — | — | — | — |
| Campesterol | 4 | 2 | 3 | — | — | 1 | 1 |
| Desmosterol | — | — | — | — | — | — | — |
| Δ⁰-Campesterol | — | — | — | — | — | — | — |
| Stigmasterol | 16 | 14 | 12 | 20 | 16 | 16 | 6 |
| Δ⁰-Stigmasterol | 1 | — | — | tr. | — | — | — |
| Sitosterol | 15 | 9 | 12 | 10 | 8 | 16 | 6 |
| Δ⁰-Sitosterol | 1 | tr. | — | — | — | — | — |
| Isofucosterol | 4 | 2 | 2 | 2 | 2 | 1 | 1 |
| Cycloartenol | 26 | 41 | 36 | 40 | 44 | 41 | 41 |
| 24-CH₂-Cycloartenol | 1 | 3 | 3 | 4 | 4 | 4 | 4 |
| 24-CH₂-Lophenol | 2 | 3 | 2 | tr. | 4 | 6 | tr. |
| Obtusifoliol | 1 | 1 | 1 | tr. | 2 | 3 | tr. |

—dash lines means not detected; tr. is trace; N.D. - not determined. NSF was chromatographed on TLC plates and bands matching 4-desmethyl-, 4-monomethyl and 4,4-dimethyl sterol standards were eluted from the plate and examined further by chromatography on 3% SE-30 columns and GC-MS. Limit of detection is 0.1 mg sterol per leaf sample.

TABLE 9
Sterol composition of plants from line G5

| Plant | G51 | G52 | G53 | G54 | G56 | G57 | G58 | G59 | G510 |
|---|---|---|---|---|---|---|---|---|---|
| Sterol | | | | | | | | | |
| Cholesterol | 13 | 5 | 6 | 11 | 16 | 11 | 4 | 15 | 5 |
| Δ⁰-Cholesterol | 1 | 1 | 1 | 1 | 1 | tr. | tr. | 1 | 1 |
| 14-α-CH₃-Δ⁷-Cholesterol | 1 | 3 | 1 | 2 | 6 | 5 | 2 | 4 | 1 |
| Δ⁷-Cholesterol | — | — | — | — | — | — | — | — | — |
| 14-α-CH₃-Δ⁸-cholesterol | — | 1 | tr. | tr. | 1 | tr. | tr. | 1 | 1 |
| Zymosterol | — | — | — | — | — | — | — | — | — |
| Δ^{7,24}-Zymosterol | — | — | — | — | — | — | — | — | — |
| 24-CH₂-Cholesterol | — | — | 3 | 4 | — | 1 | 6 | 6 | — |
| Campesterol | 8 | 15 | 4 | 2 | 1 | 2 | 2 | 3 | 19 |
| Desmosterol | — | — | — | — | 2 | — | — | — | — |
| Δ⁰-Campesterol | — | 1 | — | — | — | — | — | — | 1 |
| Stigmasterol | 20 | 6 | 10 | 13 | 20 | 17 | 4 | 11 | 6 |
| Δ⁰-Stigmasterol | — | — | tr. | tr. | — | tr. | — | — | 1 |
| Sitosterol | 21 | 11 | 7 | 9 | 9 | 8 | 3 | 11 | 1 |
| Δ⁰-Sitosterol | — | tr. | 1 | tr. | — | tr. | tr. | 1 | 1 |
| Isofucosterol | 1 | 1 | 1 | 1 | 1 | 8 | 1 | 2 | 1 |
| Cycloartenol | 34 | 48 | 58 | 52 | 41 | 47 | 49 | 35 | 43 |
| 24-CH₂-Cycloartenol | 1 | 4 | 6 | 5 | 1 | 1 | 28 | 10 | 14 |
| 24-CH₂-Lophenol | 1 | 3 | 1 | tr. | — | tr. | 1 | tr. | 4 |
| Obtusifoliol | tr. | 1 | 1 | tr. | — | tr. | tr. | tr. | 1 |

—dash lines means not detected; tr. is trace; N.D. - not determined. NSF was chromatographed on TLC plates and bands matching 4-desmethyl-, 4-monomethyl and 4,4-dimethyl sterol standards were eluted from the plate and examined further by chromatography on 3% SE-30 columns and GC-MS. Limit of detection is 0.1 mg sterol per leaf sample.

TABLE 10
Sterol composition of plants from line G6

| Plant | G63 | G65 | G66 | G67 | G68 | G69 | G610 |
|---|---|---|---|---|---|---|---|
| Sterol | | | | | | | |
| Cholesterol | 7 | 7 | 9 | 8 | 5 | 6 | 7 |
| Δ⁰-Cholesterol | tr. | 1 | 1 | 1 | tr. | tr. | 1 |
| 14-α-CH₃-Δ⁷- | 2 | 2 | 5 | 1 | 1 | 3 | 1 |
| Cholesterol | | | | | | | |
| Δ⁷-Cholesterol | — | — | — | — | — | — | — |
| 14-α-CH₃-Δ⁸- | 1 | 1 | 1 | 1 | tr. | 1 | 1 |
| cholesterol | | | | | | | |
| Zymosterol | — | — | — | — | — | — | — |
| Δ^{7,24-Zymosterol} | — | — | — | — | — | — | — |
| 24-CH₂-Cholesterol | 2 | tr. | tr. | — | — | — | — |
| Campesterol | 18 | 3 | 1 | 3 | 20 | 1 | 3 |
| Desmosterol | — | — | — | — | — | — | — |
| Δ⁰-Campesterol | tr. | — | tr. | tr. | 1 | — | — |
| Stigmasterol | 10 | 7 | 11 | 8 | 5 | 6 | 7 |
| Δ⁰-Stigmasterol | tr. | tr. | 1 | tr. | tr. | tr. | tr. |
| Sitosterol | 13 | 7 | 7 | 9 | 8 | 4 | 7 |
| Δ⁰-Sitosterol | tr. | 1 | tr. | 1 | tr. | Tr | tr. |
| Isofucosterol | 2 | 2 | 1 | 1 | 1 | tr. | 2 |
| Cycloartenol | 30 | 61 | 61 | 61 | 39 | 72 | 70 |
| 24-CH₂-Cycloartenol | 12 | 8 | 3 | 6 | 20 | 7 | 1 |
| 24-CH₂-Lophenol | 2 | — | — | — | — | — | — |
| Obtusifoliol | 1 | — | — | — | — | — | — |

—dash lines means not detected; tr. is trace; N.D. - not determined. NSF was chromatographed on TLC plates and bands matching 4-desmethyl-, 4-monomethyl and 4,4-dimethyl sterol standards were eluted from the plate and examined further by chromatography on 3% SE-30 columns and GC-MS. Limit of detection is 0.1 mg sterol per leaf sample.

These analyses indicate that cycloartenol levels of many of the transgenic plants are significantly elevated compared to controls. The cycloartenol levels achievable by this approach are at or above the level of nonutilizable sterol necessary to have a detrimental effect on insects, as demonstrated in Example 10 below. In addition, the results are consistent with successful in vivo suppression of the first methylation catalyzed by SMT.

### Example 10

### Sterol Utilization and Metabolism by Heliothis zea

Several sterols were isolated from nature or prepared synthetically to feed to the insects. An in vivo model was used involving Heliothis zea, cultured on a synthetic medium that was devoid of sterol, except for the test sterol added to the diet. Cycloartenol and several 24-methyl and -ethyl sterol isomers were found to inhibit insect growth in this in vivo model (Nes et al., 1997).

Two important sterols from corn, 24-methyl cholesta-5,23-dienol and 24-methyl cholesta-5,25(27)-dienol, were found to be non-utilizable. The 9,19-cyclopropyl sterol was also non-utilizable, as were the Δ²³⁽²⁴⁾- and Δ²⁵⁽²⁷⁾-24-alkene sterol isomers.

Heliothis zea (corn earworm) was reared on an artificial diet treated with different sterol supplements to study the relation between sterol structure and utilization in insects. H. zea eggs were used to establish a disease-free stock colony.

The stock insects were reared using sterile procedures on a pinto bean-based diet. Moths were fed 10% sucrose. Cultures were maintained at 27±1° C., at 40±10% relative humidity on a 14:10 light-dark photoperiod and an artificial diet was used to rear the insects on different sterol supplements. The experimental diet contained agar, which is known to contain trace contamination of cholesterol, otherwise the experimental diet was sterol-free.

Sterols were solubilized in acetone. Aliquots of the solutions were added to the sterol-free diet in a mortar, the material mixed thoroughly with the diet, and the organic solvent allowed to evaporate. Sterols were supplied to the medium at 200 ppm (equivalent to 1 mg of sterol per experimental vessel containing one insect).

By day 20, H. zea larva are in the final stage of larval development (sixth instar), after which the insects may pupate. A single neonate larva was placed in an experimental culture vial and allowed to grow for 20 days. The fresh weight, length and instar stage of 20-day larva were recorded.

In some treatments, the larvae were allowed to grow for another 4 days to determine whether they could pupate properly and develop into moth forms. Neonate larvae of H. zea failed to molt to the second instar when sterol was absent from the diet. Some of these insects survived for more than 15 days.

Sterols isolated from the nonsaponifiable lipid fraction extracted from larvae contain long chain fatty alcohols. These fatty alcohols may comigrate with sterols during some forms of chromatography and interfere with sterol quantitation, particularly of cholesterol. Therefore, in order to confirm the identity and amount of cholesterol in the insect an aliquot of the NSF was injected into a HPLC column and the fraction corresponding to cholesterol was examined by GC-MS.

Larvae did not develop on a sterol-less medium. Δ⁵-sterols substituted at C-24 in the side chain with hydrogen, methylene, E- or Z-ethylidene, or a- or b-ethyl groups, cholesterol, 24(28)-methylenecholesterol, sitosterol, isofucosterol, fucosterol, clinonasterol, and stigmasterol supported larval growth to late-sixth instar. These sterols are referred to as “utilizable” sterols (Table 11 and FIG. 9). In each of the incubations, the major sterol recovered from the larvae was cholesterol, showing that H. zea operates a typical insect 24-dealkylation sterol pathway.

In contrast, the sterol requirement of H. zea could not be met satisfactorily by derivatives of 3β-cholestanol with a 9β,19-cyclopropyl group, geminal dimethyl group at C-4 (e.g., cycloartenol and lanosterol), Δ⁸-bond, or by side chain modified derivatives that contained the following structural features: Δ²³⁽²⁴⁾-24-methyl or 24-ethyl group, Δ²⁴⁽²⁵⁾-24-methyl or 24-ethyl group, or Δ²⁵⁽²⁷⁾-24β-ethyl group. These are referred to as “nonutilizable” sterols (Table 11 and FIG. 9).

The major sterol recovered from larvae which developed on nonutilizable sterols was the test sterol added to the medium. Competition experiments using different proportions of cholesterol and 24, 25-dihydrolanosterol (from 9/1 to 1/9 sterol mixtures) indicated that abnormal development of H. zea may be induced on <1 to 1 sterol mixtures of utilizable and nonutilizable compounds (Table 12). Sterol absorption was related to the degree of sterol utilization and metabolism.

TABLE 11
Effect of sterols on growth and metabolism by Heliothis zea

| | | | | Total | Sterol |
|---|---|---|---|---|---|
| | | | Instar | sterol | composition³ |
| Sterol | Entry | Growth | reached by | mg/ | (as % total |
| supplement | No.¹ | response² | day 20 | insect | sterol) |
| Utilizable | | | | | |
| sterols | | | | | |
| Cholesterol | 1 | 100 | 6 | 56 | cholesterol |
| 24(28)- | 2 | 100 | 6 | 59 | ts/cholesterol |
| Methylene- | | | | | (16/84) |
| cholesterol | | | | | |
| Fucosterol | 4 | 100 | 6 | 71 | ts/cholesterol |
| | | | | | (10/90) |
| Isofucosterol | 3 | 100 | 6 | 52 | ts/desmosterol/ |
| | | | | | cholesterol |
| | | | | | (8/14/78) |
| Sitosterol | 5 | 100 | 6 | 66 | ts/cholesterol |
| | | | | | (20/80) |
| Clionasterol | 6 | 100 | 6 | 43 | ts/cholesterol |
| (50/50) | | | | | (14/84) |
| | | | | | (75/25) |
| Stigmasterol | 7 | 100 | 6 | 27 | ts/desmosterol/ |
| | | | | | cholesterol |
| | | | | | (15/1/84) |
| Nonutilizable | | | | | |
| sterols | | | | | |
| Cholest-8-enol | 13 | 5 | 3 | ND | ND |
| 24-Dehydro- | 14 | 5 | 3 | 0.6 | ts/cholesterol |
| pollinastanol | | | | | (86/14) |
| 24-Methyl- | 10 | 50 | 5 | 6 | ts/cholesterol |
| cholesta- | | | | | (80/20) |
| 5,23-dienol | | | | | |
| 24-Ethyl | 12 | 20 | 3 | 3 | ts/cholesterol |
| cholesta- | | | | | (86/14) |
| 5,23-dienol | | | | | |
| 24-Methyl | 9 | 5 | 3 | 1 | ts/cholesterol |
| cholesta- | | | | | (65/35) |
| 5,24-dienol | | | | | |
| 24-Ethyl | 11 | 10 | 3 | ND | ND |
| cholesta- | | | | | |
| 5,24-dienol | | | | | |
| Clereosterol | 8 | 20 | 3 | 3 | ts/cholesterol |
| | | | | | (80/20) |
| Ergosterol | 15 | 30 | 3 | 5 | ts/7-dehydro- |
| | | | | | cholesterol/ |
| | | | | | cholesterol |
| | | | | | (36/41/23) |
| Cycloartenol | 17 | 5 | 3 | ND | ND |
| Lanosterol | 16 | 5 | 3 | ND | ND |
| 24-Dihydro- | 18 | 5 | 3 | ND | ND |
| lanosterol | | | | | |
| ¹Structures of sterols are reported in FIG. 9. | | | | | |
| ²Growth on cholesterol produced larvae that at 20 days weighed, on average, 323 mg and were 30 mm long. Generally 16 to 20 insects survived on cholesterol to pupate and develop into adult moths. The growth responses on test sterols are relative to the growth on cholesterol which is normalized to 100%. 24-methyl cholesta-5,23-dienol was found to support pupations and adult moth formation. However, these insects exhibited congenital | | | | | |
| #deformities. Insects in the nonutilizable category generally weighed less than 100 mg per insect and their length ranged from 2 to 15 mm, with 6 to 12 insects alive at day 20. | | | | | |

³Sterols isolated from insect tissues, after the gut was purged of its contents, were analyzed by RP-HPLC and GC-MS.
ND Not determined.
ts Total sterol.

The most effective sterols were absorbed and incorporated into tissues from 27 to 66 mg per insect, whereas the least effective sterols were absorbed and incorporated into tissues from 0.6 to 6 mg per insect. These studies demonstrate that: (i) H. zea discriminates structural modifications in the sterol nucleus and side chain, (ii) the pathway of phytosterol dealkylation to cholesterol involves a high degree of regio- and stero-selectivity, and (iii) corn produces several of the nonutilizable sterols described herein.

TABLE 12
Utilization of 24-dihydrolanosterol (nonutilizable)
sparred with cholesterol (utilizable) by Heliothis zea

| | | | Instar | Total | Sterol |
|---|---|---|---|---|---|
| Sterol | | | reached | sterol | composition |
| mixture | Entry | Growth | by | mg/ | (as % total |
| (ratio) | No.* | response | day 20 | insect | sterol) |
| Cholesterol | 1 | 100 | 6 | 56 | cholesterol |
| (100%) | | | | | (100%) |
| Cholesterol/ | 1/18 | 100 | 6 | 45 | cholesterol/24,25- |
| 24,25-dihydro- | | | | | dihydrolanosterol |
| lanosterol | | | | | (93:7) |
| sterol (90:10) | | | | | |
| Cholesterol/ | 1/18 | 100 | 6 | 36 | cholesterol/24,25- |
| 24,25-dihydro- | | | | | dihydrolanosterol |
| lanosterol | | | | | (88:12) |
| sterol (70:30) | | | | | |
| Cholesterol/ | 1/18 | 70 | 6 | 25 | cholesterol/24,25- |
| 24,25-dihydro- | | | | | dihydrolanosterol |
| lanosterol | | | | | (75:25) |
| sterol (50:50) | | | | | |
| Cholesterol/ | 1/18 | 30 | 3 | 12 | cholesterol/24,25- |
| 24,25-dihydro- | | | | | dihydrolanosterol |
| lanosterol | | | | | (50:50) |
| sterol (30:70) | | | | | |
| Cholesterol/ | 1/18 | 10 | 3 | ND | ND |
| 24,25-dihydro- | | | | | |
| lanosterol | | | | | |
| (10:90) | | | | | |

*Indicates the structures in FIG. 9.

The minimal dietary concentration of cholesterol necessary for larvae to grow and pupate is 0.01% of the experimental diet. This level of cholesterol does not support a rapid rate of molting as did higher levels of cholesterol. However, diets of 0.015% cholesterol or more enhanced the rate of development of larvae. Therefore, a slightly higher amount of dietary sterol (0.02%) was used to insure that a non-limiting amount of sterol (alone or as a mixture) was available in the experimental diet, or no sterol was added to the diet to act as a control.

In all larvae treated with non-utilizable sterols, there were trace amounts of cholesterol that ranged from 80 to 350 nanograms of cholesterol per insect depending on the treatment. This source of cholesterol most likely results from carryover of cholesterol in the egg (we detected ca. 80 ng of cholesterol per egg) and from absorption of trace levels of cholesterol originally present in the agar.

As the insect increases in size, the insect may accumulate increasing amounts of cholesterol from the agar diet. Cholesterol obtained in this manner may serve as a precursor for ecdysteroid synthesis. The different effectiveness of the pair of isomers sitosterol/clionasterol and isofucosterol/fucosterol, in growth support and in their active metabolism to cholesterol indicates that the 24-dealkylation pathway may operate stereoselectively.

Developmental outcomes of H. zea larva that proceeded into moths were compared. One insect was reared on a utilizable (cholesterol treatment) sterol and the other insect(s) was reared on a non-utilizable (24-methyl cholesta-5,23-dienol treatment) sterol.

Most of the insects reared on non-utilizable sterols failed to develop beyond the third instar (Table 11), indicating they were ineffective cholesterol surrogates and harmful to growth and development. Some of the non-utilizable sterol treatments were found to pupate and develop into moths. However, these moths possessed incompletely developed wings and legs.

Table 11 and FIG. 9 show that the position of the double bond in the sterol side chain and nucleus is critical to sterol-controlled growth. The inability of cholest-8-enol to support growth suggests that H. zea cannot transform 9β,19-cyclopropyl sterols to Δ⁵-sterols.

Cyclopropyl sterols must pass through an Δ⁸-sterol intermediate to give rise to a Δ⁵-sterol. Blocking this process will lead to the formation of non-utilizable sterols. These results indicate for the first time that several sterols synthesized by corn should be unsuitable as sterol replacements of cholesterol.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### BIBLIOGRAPHY

Akisha et al. in: “Physiology and Biochemistry of Sterols” (G. W. Patterson and W. David Nes, Eds.) (1992) American Oil Chemists' Society Press, pp. 172-228

Armstrong et al. Field evaluation of European corn borer control in progeny of 173 transgenic corn events expressing the insecticidal protein from Bacillus thuringinesis. Crop Science (1995) vol. 35 pp. 550-557

Battraw and Hall. Histochemical analysis of CaMV 35S promoter-beta-glucuronidase gene expression in transgenic rice plants. Plant Mol. Biol. (1990) vol. 15 pp. 527-538

Bird and Ray. Manipulation of plant gene expression by antisense RNA. Biotechnology & Genetic Engineering Reviews (1991) 9:207-27

Bouhida et al. An analysis of the complete sequence of a sugarcane bacilliform virus genome infectious to banana and rice. Journal General Virology (1993) vol. 74 pp. 15-22

Bouvier-Nave et al. Eur. J. Biochem (1997) 246: 518-529

Bower and Birch. Transgenic sugarcane plants via microprojectile bombardment. Plant J. (1992) vol. 2 pp. 409-416

Bryant. Successful genetic engineering of a cereal crop species transgenic rye obtained by plasmid injection into floral tiller. Trends Biotechnol. vol. 5 pp. 60-61

Bytebier et al. T-DNA organization in tumor cultures and transgenic plants of the monocotyledon Asparagus officinalis. Proc. Natl. Acad. Sci. (1987) vol. 84 pp. 5345

Christou et al. Stable transformation of soybean callus by DNA-coated gold particles. Plant Physiol. (1988) vol. 87 pp. 671-674

Christou et al. Production of transgenic rice (Oryza staiva L.) plants from agronomically important indica and japonica varieties via electric discharge particle acceleration of exogenous DNA into immature zygotic embryos. (1991) vol. 9 pp. 957-962

Cheng et al. Efficient transformation of papaya by coat protein gene of papaya ringspot virus mediated by Agrobacterium following liquid-phase wounding of embryogenic tissues with carborundum. Plant Cell Reports (1996) vol. 16 pp. 127-132

Corey et al. Isolation of Arabidopsis thaliana gene encoding cycloartenol synthase by functional expression in a yeast mutant lacking lanosterol synthase by the use of a chromatographic screen. Proc. Nat. Acad. Sci. (1993) 90: 11628-11632

Coruzzi et al. Tissue-specific and light-regulated expression of a pea nuclear gene encoding the small subunit of ribulose-1,5-bisphosphate. EMBO J. (1984) vol. 3 pp. 1671-1679

Coste et al. Ecdysteroid biosynthesis and embryonic development are disturbed in insects (Locusta migratoria) reared on a plant diet (Triticum sativum) with a selectively modified sterol profile. Proc. Nat. Acad. Sci (1987) 84, pp. 643-647

De Kathen and Jacobsen. Agrobacterium tumefaciens mediated transformation of pisum sativum L. using binary and cointegrate vectors. Plant Cell Reports (1990) vol. 9 pp. 276

Fletcher, R. A. et al., 1988, in: D. Berg et al. (Eds.) STEROL BIOSYNTHESIS INHIBITORS-PHARMACEUTICAL AND AGROCHEMICAL ASPECTS, Ellis Horwood, pp 321-331

Fraley et al. Expression of bacterial genes in plant cells. Proc. Natl. Acad. Sci. (1983) vol. 80 pp. 4803-4807

Fromm et al. UCLA Symposium on Molecular Strategies for Crop Improvement (1990) Apr. 16-22. Keystone, Colo.

Gachotte et al. The Plant Journal (1996) 9: 391-398

Gibson and Shillitoe. Ribozymes. Their fucntions and strategies for their use. Molecular Biotechnology (1997) 7(2): 125-37

Gordon-Kamm et al. Transformation of maize cells and regeneration of fertile transgenic plants. Plant Cell (1990) Vol. 2, pp. 603-618

Guo et al. Developmental Regulation of Sterol Biosynthesis in Zea mays. Lipids (1995) 30: 3, p. 203-219

Guo et al. Stereochemistry of Hydrogen Migration from C-24 to C-25 during Phytosterol Biomethylation. J. Am. Chem. Soc. (1996) 118, pp. 8507-8508

Guo et al. Phytosterol Biosynthesis: Isotope Effects Associated with Biomethylation Formation to 24-Alkene Sterol Isomers. Tetrahedron Letters (1996) 37: 38, pp. 6823

Grabenok et al. Plant Mol. Biol. (1997) 34: 891-896

Haeuser, C. et al J. Plant Physiol. (1990) 137: 201-207

Hinchee et al. in PLANT CELL AND TISSUE CULTURE (1994), pp.231-271, Vasil and Thorpe (eds.), Kluwer Academic Publishers

Horn et al. Transgenic plants of orchardgrass Dactylis glomerata L. from protoplasts. Plant Cell Reports (1988) vol. 7 pp. 469-472

Husselstein et al. Transformation of Saccharomyces cerevisiae with CDNA encoding sterol c-methyl transferase from Arabidopsis thaliana results in the synthesis of 24-ethyl sterols. FEBS Letters (1996) 381: 87-92

Koziel et al. Transgenic maize plants for the control of European corn borer. Abstract, ACS annual meeting (1993) Chicago, Ill., Aug 22-27

Li, S. Stereochemical Studies on the Metabolism of Sterols by S. Cervesiae. M. S. Thesis, Texas Tech University (1996) pp. 1-123

Medberry and Olszewski. Identification of cis elements involved in commelina yellow mottle virus promoter activity. Plant Journal (1993) vol. 3 pp. 619-626

Moffatt et al. The adenine phosphoribosyltransferase-encoding gene of Arabidopsis thaliana. Gene (1994) vol 143 pp. 211-216

Nes et al. The Role of Steroids and Triterpenoids in the Growth and Reproduction of Phytophthora cactorum Lipids (1982) 17, pp. 178-183

Nes et al. Structural Requirements for Transformation of Substrates by the (S)-Adenosyl-L-methionine: Δ²⁴⁽²⁵⁾-Sterol Methyl Transferase. J. Bio. Chem. (1991) 266: 23, pp. 15202-15212

Nes et al. The Structural Requirements of Sterols for Membrane Function in Sarcharomyces cerevisiae. Arch. Biochem Biophys. (1993) 300, pp. 724-733

Nes et al. Sterol Utilization and Metabolism by Heliothis zea. Lipids (1997) 32, pp. 1317

Nes et al. Sterol Phylogenesis and Algac Evolution. Proc. Natl. Acad. Sci. (1990) 87, pp. 7565-7569

Nes et al. Overexpression, Purification and Stereochemical Studies of the Recombinant (5)-Adenosyl-L-methionine: Δ²⁴⁽²⁵⁾- to Δ²⁴⁽²⁸⁾-Sterol Methyl Transferase enzyme from Sarchoromyces cerevisiae. Arch. Biochem. Biophys. (1998) 353, pp. 01-15

Nes, W. D. and Venkatramesh, M. Enzymology of Phytosterol Transformations in “Biochemistry and Function of Sterols”, Parish, E. J. and Nes, W. D. CRS Press, Boca Raton, pp. 111-122

Ni et al. Strength and tissue specificity of chimeric promoters derived from the octopine and mannopine synthase genes. Plant J. (1995) vol. 7 pp. 661-676

Odell et al. Identification of DNA sequences required for activity of the cauliflower mosaic virus 35S promoter. Nature (1985) vol. 313 pp. 810-812

Ritchie and Hodges, in: TRANSGENIC PLANTS (1993), Vol.1, pp.147-178, Shain-dow Kung (Ed.), Academic Press Inc.

Rhodes et al. Science (1988) vol. 240 p. 204

Sanger et al. Characteristics of a strong promoter from figwort mosaic virus: comparison with the analogous 35S promoter from cauliflower mosaic virus and the regulated mannopine synthase promoter. Plant Mol. Biol. (1990) vol. 14 pp. 433-443

Sambrook, J., E. F. Fritsch, and T. Maniatis (1989) Molecular Cloning: A Laboratory Manual, Second Edition, Cold Spring Harbor Laboratory, Cold Spring Harbor, N.Y.

Schroeder et al. Transformation and regeneration of two cultivars of pea Pisum sativum L. Plant Physiol. (1993) vol. 101 pp. 751-757

Schuch, W. Using antisense RNA to study gene function. Symposia of the Society for Experimental Biology (1991) 45:117-27

Schuler et al. Nucleic Acids Res. (1982) vol. 10 pp. 8225-8244

Shi et al. Identification and Characterization of an S-adenosyl-L-Methionine Δ²⁴ -Sterol-C-methyltransferase CDNA from Soybean. J. Bio. Chem. (1996) 271: 9384-9389

Somerset al. Bio/Technology (1992) vol. 10 pp. 1589-1594

Steponkus, P. L. Annu. Rev. Plant Physiol. (1984) 35: 543-584

Tong et al. Tetrahedron Letters (1997) 38: 35, pp. 6115-6118

Toriyama et al. Bio/Technology (1988) vol. 6 p. 10

Vasil et al. Bio/Technology (1992) vol. 10 pp. 667-674

Venkatramesh et al. Mechanism and Structural Requirements for Transformation of Substrates by the (S)-adenosyl-L-methionine: Δ²⁴⁽²⁵⁾-Sterol Methyl Transferase from Saccharomyces cerevisiae. Biochimica Biophysica Acta 1299 (1996) p. 313-324

Venkatramesh et al. Sterol Specificity of the Sacchoromyces Cerevisiae ERG6 gene product Expressed in Escherichia coli Lipids (1996) 31, pp. 373-377.

Wan and Lemaux Plant Physiol. (1994) vol. 104 pp. 37-48

Wang et al. Bio/Technology (1992) vol. 10 p. 691

Weeks et al. Plant Physiol (1993) vol. 102 pp. 1077-1084

Xu et al. Rice triosephosphate isomerase gene 5′ sequence directs beta-glucuronidase activity in transgenic tobacco but requires an intron for expression in rice. Plant Physiology (1994) vol. 106 pp. 459-467

Yang et al., Plant Cell Reports (1996) 15:459-464

Zhong et al. Analysis of the functional activity of the 1.4 kb 5′ region of the rice actin 1 gene in stable transgenic plants of maize (Zea mays L.). Plant Science (1996) vol. 116 pp. 73-84

What is claimed is:

1. A double stranded DNA molecule comprising:
a promoter which functions in plants to cause the production of an RNA sequence, operably linked to
a DNA coding sequence which encodes an enzyme which binds a first sterol and produces a second sterol, operably linked to
a 3′ non-translated region which causes the polyadenylation of the 3′ end of the RNA sequence; wherein the promoter is heterologous with respect to the DNA sequence.

2. The DNA molecule of claim 1, wherein the DNA coding sequence is in the sense orientation.

3. The DNA molecule of claim 1, wherein the DNA coding sequence is in the antisense orientation.

4. The DNA molecule of claim 1, wherein the first sterol is selected from the group consisting of 4-methyl sterol, 9β,19-cyclopropyl sterol, Δ⁸-sterol, 14α-methyl sterol, Δ²³,24-alkyl sterol, Δ²⁴,24-alkyl sterol and Δ²⁵⁽²⁷⁾,24-alkyl sterol.

5. The DNA molecule of claim 1, wherein the first or second sterol lacks a Δ⁵ group.

6. The DNA molecule of claim 1, wherein the DNA coding sequence encodes an enzyme selected from the group consisting of a S-adenosyl-L-methionine-Δ²⁴⁽²⁵⁾-sterol methyl transferase, a C-4 demethylase, a cycloeucalenol to obtusifoliol-isomerase, a 14α-methyl demethylase, a Δ⁸ to Δ⁷-isomerase, a Δ⁷-C-5-desaturase and a 24,25-reductase.

7. The DNA molecule of claim 1, wherein the DNA coding sequence encodes an S-adenosyl-L-methionine-Δ²⁴⁽²⁵⁾-sterol methyl transferase (SMT).

8. The DNA according to claim 7, wherein the SMT is from plants or yeast.

9. The DNA according to claim 7, wherein the SMT is derived from Zea mays, Arabidopsis thaliana or Prototheca wickerhamii.

10. The DNA according to claim 7, wherein the SMT is yeast ERG6.

11. A transgenic plant comprising a double stranded DNA molecule comprising:
a promoter which functions in plants to cause the production of an RNA sequence, operably linked to
a DNA coding sequence which encodes an enzyme which binds a first sterol and produces a second sterol, operably linked to
a 3′ non-translated region which causes the polyadenylation of the 3′ end of the RNA sequence; wherein the promoter is heterologous with respect to the DNA sequence.

12. The plant of claim 11, wherein the DNA coding sequence is in the sense orientation.

13. The plant of claim 11, wherein the DNA coding sequence is in the antisense orientation.

14. The plant of claim 11, wherein the first sterol is selected from the group consisting of 4-methyl sterol, 9β,19-cyclopropyl sterol, Δ⁸-sterol, 14α-methyl sterol, Δ²³,24-alkyl sterol, Δ²⁴,24-alkyl sterol and Δ²⁵⁽²⁷⁾,24-alkyl sterol.

15. The plant of claim 11, wherein the first or second sterol lacks a Δ⁵ group.

16. The plant of claim 11, wherein the DNA coding sequence encodes an enzyme selected from the group consisting of a S-adenosyl-L-methionine-Δ²⁴⁽²⁵⁾-sterol methyl transferase, a C-4 demethylase, a cycloeucalenol to obtusifoliol-isomerase, a 14α-methyl demethylase, a Δ⁸ to Δ⁷-isomerase, a Δ⁷-C-5-desaturase and a 24,25-reductase.

17. The plant of claim 11, wherein the DNA coding sequence encodes an S-adenosyl-L-methionine-Δ²⁴⁽²⁵⁾-sterol methyl transferase (SMT).

18. The plant of claim 17, wherein the SMT is from plants or yeast.

19. The plant of claim 17, wherein the SMT is derived from Zea mays, Arabidopsis thaliana or Prototheca wickerhamii.

20. The plant of claim 17, wherein the SMT is yeast ERG6.

21. The plant of claim 11, which plant is resistant to an insect, nematode or pythiaceous fungus.

22. The plant of claim 11, which plant has an increased level of a cholesterol-reducing sterol.

23. The plant of claim 22, wherein the sterol is cycloartenol or sitosterol.

24. The plant according to claim 11, which plant is resistant to drought, salinity or severe cold.

25. The plant according to claim 11, which plant is a tomato, corn or soybean plant.

26. A process of producing a transgenic plant comprising:
(a) transforming plant cells with a recombinant DNA molecule comprising:
a promoter which functions in plants to cause the production of an RNA sequence, operably linked to
a DNA coding sequence which encodes an enzyme which binds a first sterol and produces a second sterol, operably linked to
a 3′ non-translated region which causes the polyadenylation of the 3′ end of the RNA sequence; wherein the promoter is heterologous with respect to the DNA sequence;
(b) selecting transformed plant cells comprising the recombinant DNA molecule; and
(c) regenerating transgenic plants from the transformed plant cells.

27. The process of claim 26, wherein the DNA coding sequence is in the sense orientation.

28. The process of claim 26, wherein the DNA coding sequence is in the antisense orientation.

29. The process of claim 26, wherein the first sterol is selected from the group consisting of 4-methyl sterol, 9β,19-cyclopropyl sterol, Δ⁸-sterol, 14α-methyl sterol, Δ²³,24-alkyl sterol, Δ²⁴,24-alkyl sterol and Δ²⁵⁽²⁷⁾,24-alkyl sterol.

30. The process of claim 26, wherein the first or second sterol lacks a Δ⁵ group.

31. The process of claim 26, wherein the DNA coding sequence encodes an enzyme selected from the group consisting of a S-adenosyl-L-methionine-Δ²⁴⁽²⁵⁾-sterol methyl transferase, a C-4 demethylase, a cycloeucalenol to obtusifoliol-isomerase, a 14α-methyl demethylase, a Δ⁸ to Δ⁷-isomerase, a Δ⁷-C-5-desaturase and a 24,25-reductase.

32. The process of claim 26, wherein the DNA coding sequence encodes an S-adenosyl-L-methionine-Δ²⁴⁽²⁵⁾-sterol methyl transferase (SMT).

33. The process of claim 32, wherein the SMT is from plants or yeast.

34. The process of claim 32, wherein the SMT is derived from Zea mays, Arabidopsis thaliana or Prototheca wickerhamii.

35. The process of claim 32, wherein the SMT is yeast ERG6.

36. The process of claim 26, wherein the plant is resistant to an insect, nematode or pythiaceous fungus.

37. The process of claim 26, wherein the plant has an increased level of a cholesterol-reducing sterol.

38. The process of claim 37, wherein the sterol is cycloartenol or sitosterol.

39. The process of claim 26, wherein the plant is resistant to drought, salinity or severe cold.

40. The process of claim 26, wherein the plant is a tomato, corn or soybean plant.


SEQUENCE LISTING (16 sequences)

SEQ ID NO: 1 - DNA, 1320 bp, Saccharomyces cerevisiae
ttactttcgatttaagttttacataatttaaaaaaacaagaataaaataataatatagtaggcagcataagatgagtgaaacagaattgagaaaaagacaggcccaattcactagggagttacatggtgatgatattggtaaaaagacaggtttgagtgcattgatgtcgaagaacaactctgcccaaaaggaagccgttcagaagtacttgagaaattgggatggtagaaccgataaagatgccgaagaacgtcgtcttgaggattataatgaagccacacattcctactataacgtcgttacagatttctatgaatatggttggggttcctctttccatttcagcagattttataaaggtgagagtttcgctgcctcgatagcaagacatgaacattatttagcttacaaggctggtattcaaagaggcgatttagttctcgacgttggttgtggtgttgggggcccagcaagagagattgcaagatttaccggttgtaacgtcatcggtctaaacaataacgattaccaaattgccaaggcaaaatattacgctaaaaaatacaatttgagtgaccaaatggactttgtaaagggtgatttcatgaaaatggatttcgaagaaaacactttcgacaaagtttatgcaattgaggccacatgtcacgctccaaaattagaaggtgtatacagcgaaatctacaaggttttgaaaccgggtggtacctttgctgtttacgaatgggtaatgactgataaatatgacgaaaacaatcctgaacatagaaagatcgcttatgaaattgaactaggtgatggtatcccaaagatgttccatgtcgacgtggctaggaaagcattgaagaactgtggtttcgaagtcctcgttagcgaagacctggcggacaatgatgatgaaatcccttggtattacccattaactggtgagtggaagtacgttcaaaacttagctaatttggccacatttttcagaacttcttacttgggtagacaatttactacagcaatggttactgtaatggaaaaattaggtctagccccagaaggttccaaggaagttactgctgctctagaaaatgctgcggttggtttagttgccggtggtaagtccaagttattcactccaatgatgcttttcgtcgctaggaagccagaaaacgccgaaaccccctcccaaacttcccaagaagcaactcaataaattcactagatcaataagattcaaataaagcgcacgatatatacctattttcctatatatgcagataaaaagatagcacgttcattgctagcaggcc

SEQ ID NO: 2 - protein, 383 aa, Saccharomyces cerevisiae
MSETELRKRQAQFTRELHGDDIGKKTGLSALMSKNNSAQKEAVQKYLRNWDGRTDKDAEERRLEDYNEATHSYYNVVTDFYEYGWGSSFHFSRFYKGESFAASIARHEHYLAYKAGIQRGDLVLDVGCGVGGPAREIARFTGCNVIGLNNNDYQIAKAKYYAKKYNLSDQMDFVKGDFMKMDFEENTFDKVYAIEATCHAPKLEGVYSEIYKVLKPGGTFAVYEWVMTDKYDENNPEHRKIAYEIELGDGIPKMFHVDVARKALKNCGFEVLVSEDLADNDDEIPWYYPLTGEWKYVQNLANLATFFRTSYLGRQFTTAMVTVMEKLGLAPEGSKEVTAALENAAVGLVAGGKSKLFTPMMLFVARKPENAETPSQTSQEATQ

SEQ ID NO: 3 - DNA, 1420 bp, Arabidopsis thaliana
ctctctctctctctctcttggtcttcctcactcttaacgaaaatggactctttaacactcttcttcaccggtgcactcgtcgccgtcggtatctactggttcctctgcgttctcggtccagcagagcgtaaaggcaaacgagccgtagatctctctggtggctcaatctccgccgagaaagtccaagacaactacaaacagtactggtctttcttccgccgtccaaaagaaatcgaaaccgccgagaaagttccagacttcgtcgacacattctacaatctcgtcaccgacatatacgagtggggatggggacaatccttccacttctcaccatcaatccccggaaaatctcacaaagacgccacgcgcctccacgaagagatggcggtagatctgatccaagtcaaacctggtcaaaagatcctagacgtcggatgcggtgtcggcggtccgatgcgagcgattgcatctcactcgcgagcaacgtagtcgggattacaataaacgagtatcaggtgaacagagctcgtctccacaataagaaagctggtctcgacgcgctttgcgaggtcgtgtgtggtaacttcctccagatgccgttcgatgacaacagtttcgacggagcttattccatcgaagccacgtgtcacgcgccgaagctggaagaagtgtacgcagagatctacagggtgttgaaacccggatctatgtatgtgtcgtacgagtgggttacgacggagaaatttaaggcggaggatgacgaacacgtggaggtaatccaagggattgagagaggcgatgcgttaccagggcttagggcttacgtggatatagctgagacggctaaaaaggttgggtttgagatagtgaaggagaaggatctggcgagtccaccggctgagccgtggtggactaggcttaagatgggtaggcttgcttattggaggaatcacattgtggttcagattttgtcagcggttggagttgctcctaaaggaactgttgatgttcatgagatgttgtttaagactgctgattgtttgaccagaggaggtgaaaccggaatattctctccgatgcatatgattctctgcagaaaaccggagtcaccggaggagagttcttgagaaaggtagaaaggaaacatcaccggaaaaagtatggagaattttctcaatttgtttttatttttaagttaaatcaacttggttattgtactatttttgtgttttaatttggtttgtgtttcaagaattattagtttttttttgttttgttgcatatgagaatcttactcttgatttctccgccgtagagccggcgagacataggggattattagtatttttaagtgtgtttaagattgattaacaagttagtaaaataaaatgtacttaggtgtcgaaaaaaaaaggaattc

SEQ ID NO: 4 - protein, 361 aa, Arabidopsis thaliana
MDSLTLFFTGALVAVGIYWFLCVLGPAERKGKRAVDLSGGSISAEKVQDNYKQYWSFFRRPKEIETAEKVPDFVDTFYNLVTDIYEWGWGQSFHFSPSIPGKSHKDATRLHEEMAVDLIQVKPGQKILDVGCGVGGPMRAIASHSRANVVGITINEYQVNRARLHNKKAGLDALCEVVCGNFLQMPFDDNSFDGAYSIEATCHAPKLEEVYAEIYRVLKPGSMYVSYEWVTTEKFKAEDDEHVEVIQGIERGDALPGLRAYVDIAETAKKVGFEIVKEKDLASPPAEPWWTRLKMGRLAYWRNHIVVQILSAVGVAPKGTVDVHEMLFKTADCLTRGGETGIFSPMHMILCRKPESPEESS

SEQ ID NO: 5 - DNA, 1320 bp, Saccharomyces cerevisiae; identical to SEQ ID NO: 1

SEQ ID NO: 6 - DNA, 1497 bp, Zea mays
agactctggttctgacatgcagcaattattgcaggtgcatttgatccgtcccggccgcctacacgatgtccaagtcgggagcgctggatcttgcttctggcctcggagggaagatcaacaaggtggaagtcaagtcggccgtcgatgagtatgagaaatatcatggatactatggagggaaggaggaagcaaggaagtccaactatactgatatggttaataaatactatgatcttgccactagcttctatgagtatggttggggtgaatccttccactttgctcacagatggaatggagaatccttacgtgaaagcatcaagcgacatgagcattttcttgccctgcaacttggtttgaaaccaggaatgaaggttttagatgtgggctgtggaataggtggaccactgagagaaattgcaagatttagctcaacttcagttaccggattgaataacaacgaataccagataaccaggggaaaggagctcaaccgtttagcaggaattagtggaacatgtgattttgtcaaggcggacttcatgaagatgccgttcgatgacaacacttttgatgctgtttacgccattgaggcaacatgtcatgcacctgatccagttggttgctacaaggagatatatcgtgtgttgaagcctggccagtgctttgccgtgtacgagtggtgcgttacggatcactatgatcctaacaatgcaacccacaaaaggatcaaggatgaaattgagcttggcaatggcctgccagatatcagaagcactcggcaatgtctccgggcagtaaaagacgccgggtttgaggttgtttgggataaggatcttgctgaagattctcccttgccttggtacttgcccttggatccaagccgattctccctgagtagcttccgtttgacctctgtgggacgcatgattacccgcacaatggtcaaggccctggagtacgttggtcttgctccgcagggcagtgagagggtctctagtttcctggagaaggctgcagaagggctggtagagggcggaaagaaggagatcttcacgccaatgtacttcttttttgttcggaagcctcttctggaatgagctcttggatcaccttttcagagagagaaggcaagtggtcatttcgaagaagccgaggagagggaacctggaatcaagaaaaccttcagctctcctgtgtaggaggaaagttaacgaacagtgtagtaactgttcagctctgtgtttattcagttgttttgctgcttgaggttattcgtttctaggtgggggttggaatccttttcgccataaacctctcagtggcataaataagatggtttgcataagagtacttcatggataccgtaagggctactactgaaagagaaatgtttaagcagcatggtatgtgagcaactagtgataattattccatccttttttttaatataaagcaggagttttgtcaaaaaaaaaaaaaaaaaaaaaaaaaaaa

SEQ ID NO: 7 - protein, 344 aa, Zea mays
MSKSGALDLASGLGGKINKVEVKSAVDEYEKYHGYYGGKEEARKSNYTDMVNKYYDLATSFYEYGWGESFHFAHRWNGESLRESIKRHEHFLALQLGLKPGMKVLDVGCGIGGPLREIARFSSTSVTGLNNNEYQITRGKELNRLAGISGTCDFVKADFMKMPFDDNTFDAVYAIEATCHAPDPVGCYKEIYRVLKPGQCFAVYEWCVTDHYDPNNATHKRIKDEIELGNGLPDIRSTRQCLRAVKDAGFEVVWDKDLAEDSPLPWYLPLDPSRFSLSSFRLTSVGRMITRTMVKALEYVGLAPQGSERVSSFLEKAAEGLVEGGKKEIFTPMYFFFVRKPLLE

SEQ ID NO: 8 - protein, 6 aa, Artificial Sequence
  Synthetic Peptide
YEYGWG

SEQ ID NO: 9 - protein, 6 aa, Artificial Sequence
  Synthetic Peptide
GCGVGG

SEQ ID NO: 10 - protein, 6 aa, Artificial Sequence
  Synthetic Peptide
ATCHAP

SEQ ID NO: 11 - protein, 6 aa, Artificial Sequence
  Synthetic Peptide
EWVMTD

SEQ ID NO: 12 - DNA, 17 bp, Artificial Sequence
  Synthetic primer
taygartdkgghtgggg

SEQ ID NO: 13 - DNA, 17 bp, Artificial Sequence
  Synthetic primer
ggatgyggwrtyggsgg

SEQ ID NO: 14 - DNA, 17 bp, Artificial Sequence
  Synthetic primer
gccacdtgycaygcdcc

SEQ ID NO: 15 - DNA, 17 bp, Artificial Sequence
  Synthetic primer
tcvgtcrtdmmccaytc

SEQ ID NO: 16 - DNA, 13 bp, Artificial Sequence
  Synthetic primer
gggctgcagtggc